(19) United States
(12) Patent Application Publication
(10) Pub. No.: US 2020/0061215 A1
(43) Pub. Date: Feb. 27, 2020

(54) MALTOTRIOSE-BASED PROBES FOR FLUORESCENCE AND PHOTOACOUSTIC IMAGING OF BACTERIA

(71) Applicant: The Board of Trustees of the Leland Stanford Junior University, Stanford, CA (US)

(72) Inventors: Aimen Zlitni, Mountain View, CA (US); Gayatri Gowrishankar, Cupertino, CA (US); Sanjiv S. Gambhir, Portola Valley, CA (US)

(21) Appl. No.: 16/546,936

(22) Filed: Aug. 21, 2019

Related U.S. Application Data
(60) Provisional application No. 62/721,843, filed on Aug. 23, 2018.

(51) Int. Cl. A61K 49/00; C12N 1/20; G01N 33/58

(52) U.S. Cl. CPC A61K 49/0052; C12N 1/20; G01N 2800/52; A61K 49/0032; G01N 33/582

(57) ABSTRACT

Embodiments of the present disclosure provide for labeled maltotriose probes, methods of making labeled probes, pharmaceutical compositions including labeled probes, methods of using labeled probes, methods of diagnosing, localizing, monitoring, and/or assessing bacterial infections, using labeled probes, kits for diagnosing, localizing, monitoring, and/or assessing bacterial infections, using labeled probes, and the like.

### CROSS-REFERENCE TO RELATED APPLICATIONS

This application claims priority to and the benefit of U.S. Provisional Application 62/721,843 titled “FLUORESCENCE AND PHOTOACOUSTIC IMAGING OF BACTERIAL INFECTIONS, TOWARDS A NEW GENERATION OF MALTOTRIOSE-BASED PROBES” filed Aug. 23, 2018, the entire disclosure of which is incorporated herein by reference in its entirety.

### TECHNICAL FIELD

The present disclosure is generally related to a bacteria-specific fluorescent and photoacoustic probe. The present disclosure is further related to a method of imaging a bacterial infection or colonization in a human or animal subject.

### BACKGROUND

Bacterial infections are of mounting medical and public concern worldwide. One major reason for this epidemic is the overuse of antimicrobials that lead to an increase in the number of drug-resistant bacterial strains (Fiore et al., J. Fam. Pract. 66: 730-736 (2017); McKenna, M. Nature 499: 394-396 (2013)). Furthermore, an increase in human life expectancy contributes to the high number of individuals at risk of infection and the proliferation of necessary medical procedures (i.e. surgery, arthroplasty, fracture fixations, biomedical implantations) (Sporer et al., J. Am. Acad. Orthop. Surg. 14: 246-255 (2006)). Such procedures and associated implants are susceptible to introducing infection (Edwards et al., Curr. Opin. Infect. Dis. 17: 91-96 (2004); Bode et al., N. Engl. J. Med. 362: 9-17 (2010); Arciola et al., Nat. Revs. Microbiol. 16: 397-409 (2018); Anderson, D. J. Infect. Dis. Clin. North Am. 25: 135-153 (2011)).

Wound and surgical infections cause a huge burden on patient's quality of life and are result in delayed healing and can lead to death (Anderson, D. J. Infect. Dis. Clin. North Am. 25: 135-153 (2011)). For example, surgical site infections (SSIs) are one of the most common types of healthcare-associated infections and occur in 2-5% of patients undergoing surgery in the United States. This translates to around 400,000 SSIs for an average of 15 million procedures performed annually in the US. In addition to increasing the duration of hospitalization, SSIs increase treatment cost as well as mortality risk by 2-11 fold (Anderson & Kaye Infect. Dis. Clin. North Am. 23: 53-72 (2009)).

Unfortunately, many of these infections are only diagnosed after becoming systemic or have caused damage to key organs making it harder and costly to treat due to the high bacterial burden. There is a continuing need, therefore, to develop tools to non-invasively detect bacterial infections at an early stage with high sensitivity and specificity. Such tools can aid clinicians in deciding the optimal route of treatment after surgeries and can be used to monitor the effectiveness of the treatment regimen to insure proper management of wound and surgical infections.

In the clinic, bacterial infections are diagnosed though a combination of clinical, laboratory (detecting signs of inflammation, microbiology and histopathology) and imaging assessments that can be invasive, time consuming and costly (Arciola et al., Nat. Revs. Microbiol. 16: 397-409 (2018); Anderson, D. J. Infect. Dis. Clin. North Am. 25: 135-153 (2011); Trampuz & Zimmerli Injury 37: S59-S66 (2006)). Currently, imaging modalities such as x-ray, ultrasound, magnetic resonance imaging (MRI) and computer tomography (CT) provide valuable anatomical information but are only useful in diagnosing delayed and late-stage infections (Trampuz & Zimmerli Injury 37: S59-S66 (2006). To improve sensitivity, specificity and early detection, a molecular imaging strategy that necessitates the development of imaging probes specifically targeting sites of bacterial infections will be required.

A variety of radio-imaging agents for positron emission tomography (PET) tracers for whole-body bacterial imaging have been developed and currently are under evaluation in clinical trials. However, these radio-imaging approaches require availability of nearby cyclotrons and generators for isotope production and experienced radiochemists for tracer production, thus limiting their availability on demand. Use is limited to a hospital setting for the management of in-patients. Currently there are no rapid and reliable diagnostic techniques that can detect implant, wound and surgical infections at an early stage in outpatient clinics. More specifically, a tool that can aid doctors in emergency rooms and in field hospitals to quickly diagnose bacterial wound infections and determine the extent of infection can change clinical management.

Fluorescence imaging (FLI) relies on detecting emission signals from fluorescent probes upon excitation at their appropriate absorbance wavelength (Sevick-Muraca, E. M. Annu. Rev. Med. 63: 217-231 (2012); James & Gambhir Physiol. Rev. 92: 897-965 (2012)). Fluorescence imaging of bacterial infections has gained attention due to its many advantages such as high resolution, real-time imaging capabilities, ease of use, and low cost. Constricted by its limited depth and penetration (approximately 1 cm), fluorescence imaging can only be implemented in superficial infection imaging (during surgery, of superficial implants, endoscopes and the like, and intra-operative applications) (Sevick-Muraca, E. M. Ann. Rev. Med. 63: 217-231 (2012); James & Gambhir Physiol. Rev. 92: 897-965 (2012)). On the other hand, photoacoustic imaging (PA) is an emerging imaging technique that relies on detecting ultrasound signals produced upon thermal expansion of tissue when exciting the fluorescent probe at an appropriate wavelength (Wang & Hu, S. Science 335: 1458-1462 (2012)).

Photoacoustic imaging (PA) is shown to be a standalone portable tool capable of imaging endogenous signals such as melanin or hemoglobin and exogenous chromophores with deeper imaging capabilities (up to 4 cm) than fluorescence imaging and better resolution than MRI (Wang & Hu, S. Science 335: 1458-1462 (2012); Beard, P. Interface Focus 1: 602-631 (2011); Mallidi et al., Trends in Biotechnol. 29: 213-221 (2011); Steinberg et al. Photoacoustics 14: 77-98 (2019)). In addition, photoacoustic imaging can be used to monitor tissue healing by imaging blood vessels as well as utilizing its ultrasound component to provide anatomical information (Beard, P. Interface Focus 1: 602-631 (2011); Zhang et al., Phys. Med. Biol. 54: 1035-1046 (2009); Mari et al., J Biomed Opt 20: 110503 (2015)). Thus, combined fluorescence and photoacoustic imaging, can be an optimal cost-effective and non-invasive tool to quickly detect bacterial infections and monitor effectiveness of treatment at local sites (i.e. surgery and injury sites).

A number of FLI and/or PAI probes targeted to bacteria have been developed by using antibiotics (vancomycin ((van Oosten et al., Nat. Commun. 4: 2584 (2013); Li et al., Adv. Mater. 28: 254-262 (2016)) or teicoplanin (Wang et al., J. Am. Acad. Orthop. Surg. 25 Suppl 1: S7-S12 (2017)) specific to Gram-positive bacteria), Concanavalin (targeting bacterial cell-surface mannose) (Tang et al., J. Biomed. Nanotechnol. 10: 856-863 (2014)), antibodies (targeting the immunodominant staphylococcal antigen A, specific to S. aureus) (Romero Pastrana et al., Virulence 9: 262-272 (2018)), boronic-acid (targeting bacterial cell-surface glycoproteins, specific to Gram-positive bacteria) (Kwon et al., Angew. Chem. Int. Ed. Engl. 58: 8426-8431 (2019)), enzyme-activated nanoparticles (targeting gelatinase-expressing Gram-positive bacteria) (Li et al., Adv. Mater. 28: 254-262 (2016)) or through electrostatic and hydrophobic interactions (specific to Gram-positive bacteria) (Zhou et al., J. Mat. Chem. B 4: 4855-4861 (2016)). Preclinical evaluation of these probes showed promising results in FLI (van Oosten et al., Nat. Commun. 4: 2584 (2013); Tang et al., J. Biomed. Nanotechnol. 10: 856-863 (2014); Romero Pastrana et al., Virulence 9: 262-272 (2018); Kwon et al., Angew. Chem. Int. Ed. Engl. 58: 8426-8431 (2019)) or PAI (Li et al., Adv. Mater. 28: 254-262 (2016); Wang et al., J. Am. Acad. Orthop. Surg. 25 Suppl 1: S7-S12 (2017)) of some bacterial infections. Unfortunately, targeting bacterial cell wall potentially limits the amount of signaling agent taken up leading to lower sensitivity. In addition, strain-specific probes will have minimal clinical impact in imaging surgery and injury related bacterial infections since they usually occur from the presence of a variety of pathogenic bacteria. A few other examples rely on genetically encoding bacteria with reporters, such as photo-switchable chromoproteins (Yao et al., Nat. Methods 13: 67-73 (2015); Chee et al., J. Biomed. Opt. 23: 106006 (2018)) and violacein (Jiang et al., Sci. Rep. 5: 11048 (2015)), have been reported. While these strategies allow noninvasive imaging of bacteria in vivo using photoacoustic imaging, the application of such platform would be limited to visualizing biochemistry, pathophysiological processes and gene expression profiles in living subjects as well as imaging tumor homing bacteria (Peters et al., Nat. Commun. 10: 1191 (2019)) and cannot be used for diagnosing bacterial infections.

Another bacterial-imaging strategy relies on the utility of large sugar molecules to deliver the signaling agent into bacteria. These large sugars (such as maltose, maltotriose and maltohexose) are a major source of glucose for bacteria and are taken up in millimolar quantities (Ning et al., Nat. Mater. 10: 602-607 (2011)). A major advantage of such probes is their specific uptake by bacteria though an antigen binding cassette (ABC) transporter not present in mammalian cells and which allows differentiation of bacterial infections from other diseases such as cancer and inflammation.

Murthy and coworkers developed a fluorescent (Ning et al., Nat. Mater. 10: 602-607 (2011)) and an ¹⁸F-labeled (Ning et al., Angew. Chem. Int. Ed. Engl. 53: 14096-14101 (2014)) derivative of maltohexose at the anomeric carbon and showed its effectiveness in fluorescence and PET imaging of bacterial infections in rats respectively (Takemiya et al., JACC Cardiovasc. Imaging 12; 875-886 (2019)). Recently, Pang and coworkers developed theranostic nanoparticles loaded with purpurin 18 and targeted to bacteria by surface functionalization to maltohexose (Pang et al., ACS Nano. acsnano.8b09336 (2019)). These particles showed great potential in treating bacteria using sonodynamic therapy and assessed the specificity of their particles to infection site using FLI imaging and showed an example of PAI using their particles.

Also developed was an ¹⁸F-6″-labeled maltose and maltotriose derivatives and their effectiveness was shown in imaging bacterial infections though PET imaging (Gowrishankar et al., PLoS ONE 9: e107951 (2014); Namavari et al., Mol. Imaging Biol. 17: 168-176 (2015); Gowrishankar et al., J. Nuclear Med. 58: 1679-1684 (2017)).

### SUMMARY

The present disclosure encompasses the development and evaluation of a novel fluorescent derivatives of maltotriose useful for photoacoustic and fluorescent imaging of bacterial infections.

One aspect of the disclosure encompasses embodiments of a probe comprising an oligosaccharide selectively taken up by a bacterial population and not by a mammalian cell, wherein the oligosaccharide can be connected to a detectable label by a linker and having the formula:

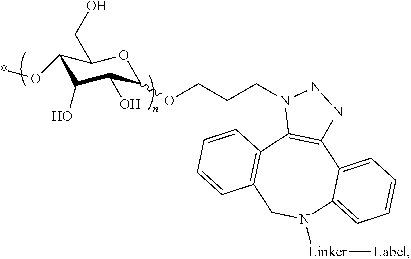

wherein n=1-8.

In some embodiments of this aspect of the disclosure, n=3 and the oligosaccharide can be a maltotriose, the probe having the formula I:

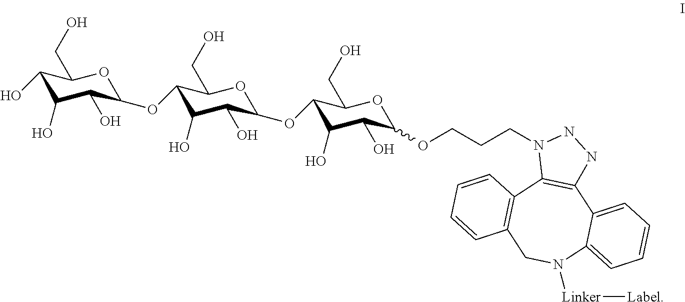

In some embodiments of this aspect of the disclosure, the detectable label can be a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

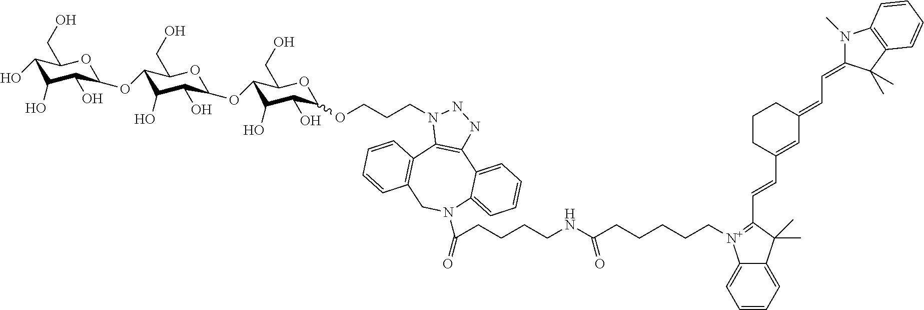

Another aspect of the disclosure encompasses embodiments of a composition comprising a probe, wherein the probe can comprise an oligosaccharide selectively taken up by a bacterial population and not by a mammalian cell and connected to a detectable label by a linker, and having the formula:

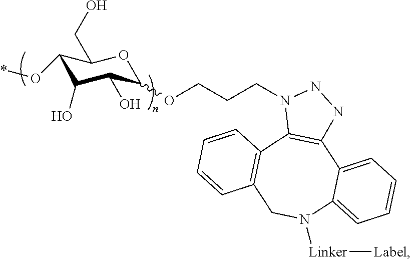

wherein n=1-8; and a pharmaceutically acceptable carrier.

In some embodiments of this aspect of the disclosure, n=3 and the oligosaccharide can be a maltotriose, the probe having the formula I:

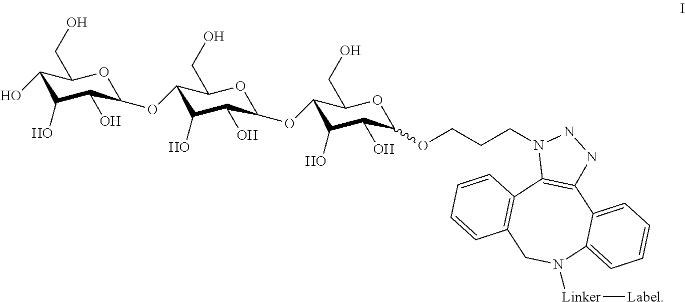

In some embodiments of this aspect of the disclosure, the detectable label is a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

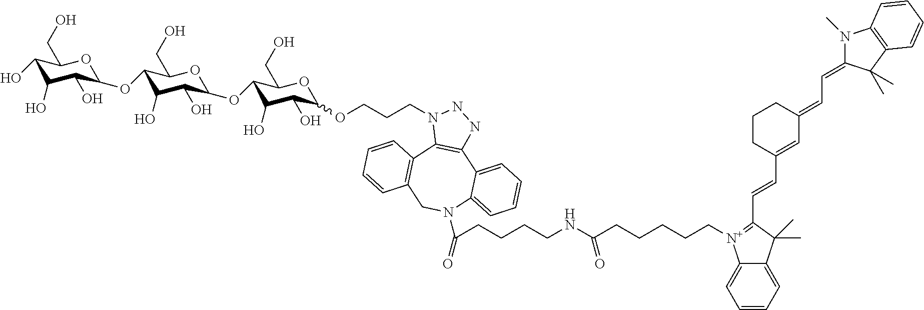

In some embodiments of this aspect of the disclosure, the composition can further comprise a therapeutic agent.

In some embodiments of this aspect of the disclosure, the therapeutic agent can be an anti-bacterial agent.

Yet another aspect of the disclosure encompasses embodiments of a method of imaging a bacterial population comprising: (i) contacting a suspected bacterial population with a composition comprising a probe, wherein the probe comprises an oligosaccharide selectively taken up by a bacterial population and not by a mammalian cell and connected to a detectable label by a linker and having the formula:

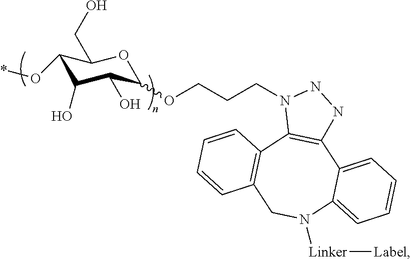

wherein n=1-8; (ii) imaging at least a portion of the subject; and (iii) detecting the labeled probe, wherein the location of the labeled probe corresponds to a bacterial population.

In some embodiments of this aspect of the disclosure, n=3 and the oligosaccharide can be a maltotriose, the probe having the formula I:

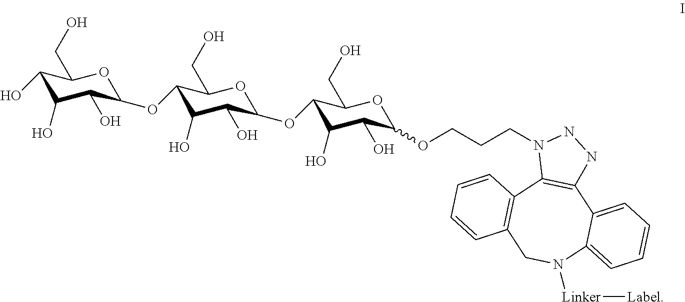

In some embodiments of this aspect of the disclosure, the detectable label can be a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

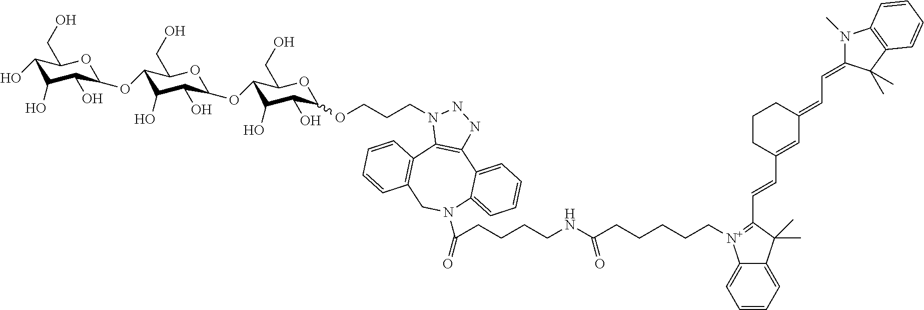

In some embodiments of this aspect of the disclosure, the method can further comprise repeating the steps (i)-(iii) periodically to monitor the progress of a bacterial infection or colonization.

In some embodiments of this aspect of the disclosure, the probe can be detected by the detection of a fluorescence signal emitted by the probe.

In some embodiments of this aspect of the disclosure, the probe can be detected by the detection of a photoacoustic signal emitted by the probe.

In some embodiments of this aspect of the disclosure, the bacterial population can be an infection of a human or animal subject.

In some embodiments of this aspect of the disclosure, the bacterial population can be a bacterial colonization of a surface.

In some embodiments of this aspect of the disclosure, the surface can be that of a surgical instrument.

In some embodiments of this aspect of the disclosure, the probe can be co-administered to the recipient subject with at least one therapeutic agent.

In some embodiments of this aspect of the disclosure, the probe can be administered to the recipient subject before administering at least one therapeutic agent.

In some embodiments of this aspect of the disclosure, the probe can be administered to the recipient subject with at least one therapeutic agent, wherein the at least one therapeutic agent is an antibiotic.

In some embodiments of this aspect of the disclosure, the method can further comprising the step of generating a series of images over a period of time, thereby indicating if the bacterial population changes in size.

Other compositions, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates the synthesis of Cy7-1-maltotriose (compound 3a, n=1) and Cy7-1-maltohexose (compound 3b, n=4). DBCO=dibenzoyl cyclooctyne; RT=room temperature; Me=CH₃; Et=CH₃CH₂; DCM: dichloromethane.

FIGS. 2A-2C illustrate in vitro evaluation of Cy7-1-maltotriose 3a and Cy7-1-maltohexose 3b.

FIG. 2A illustrates a bar graph showing the effectiveness of 1-maltotriose and 1-maltohexose derivatives in competing with the uptake of ³H-maltose in E. coli. Reduction in ³H-maltose uptake in E. coli was observed with prior incubation with any of the 1-maltotriose or 1-maltohexose derivatives compared to E. coli incubated with only ³H-maltose (P<0.0001). Data presented as counts per minute (CPM) in each sample normalized to protein content (μg of protein).

FIG. 2B illustrates a bar graph showing quantified fluorescence signal in E. coli, Staphylococcus aureus, Bacillus subtilis and Pseudomonas aeruginosa after incubation with Cy7-1-maltotriose. As a control, sodium azide-inactivated E. coli and E. coli-mutants lacking components of the maltodextrin transporter were used. Reduction in Cy7-1-maltotriose uptake in inactivated E. coli and E. coli mutations was observed (P<0.0001). All experiments were conducted in triplicate and statistical analysis was performed using one-way ANOVA.

FIG. 2C illustrates a bar plot representation of influx of Cy7-1-maltotriose and Cy7-1-maltohexose in E. coli overtime. Quantified fluorescence signal from influx study showcases significant increase in uptake of the probes when incubated for 60 min compared to 30 min incubation for both probes (P<0.0137 and P<0.0001 for Cy7-1-maltotriose and Cy7-1-maltohexose, respectively, n=3). In addition, significantly higher fluorescence uptake of Cy7-1-maltotriose was observed compared to Cy7-1-maltohexose when incubated for 30 min was observed (P<0.0001, n=3). Statistical analysis was performed using one-way ANOVA.

FIGS. 3A-3D illustrate the in vivo validation of Cy7-1-maltotriose in an E. coli-induced myositis murine model.

FIG. 3A illustrates fluorescence imaging showing the accumulation of Cy7-1-maltotriose in E. coli-infected thigh muscle as early as 1 hr post intravenous injection (right thigh muscle). There was no accumulation of the agent in the left thigh muscle injected with 10⁸ CFUs of heat-inactivated E. coli.

FIG. 3B illustrates a bar graph of quantified fluorescence signal in right and left thigh muscle. As early as 1 hr post injection, a significantly higher fluorescence signal was found in muscle infected with E. coli (right thigh) compared to muscle infected with heat-inactivated E. coli (left thigh) (n=4).

FIG. 3C illustrates a 3D-rendered photoacoustic image overlaid on an ultrasound image of a mouse left (control) and right (infected) thigh muscle pre- and 20 hr post-injection of Cy7-1-maltotriose via the tail vein. Qualitatively, the image of the infected thigh muscle (bottom right) shows a higher photoacoustic signal relative to image of the same muscle acquired before probe injection (top right) and image of the control thigh muscle (bottom left).

FIG. 3D illustrates a bar graph of the quantified photoacoustic signal intensity in the photoacoustic images acquired before and 20 hr after injection of Cy7-1-maltotriose. The quantified signal of the infected thigh muscle was higher in the images acquired post injection of the probe compared to that before injection (n=4 and 5 respectively, *P=0.0006). In addition, the signal in the infected thigh muscle post injection was higher than that of the control muscle (n=4, **P=0.0059).

FIGS. 4A-4D illustrate an in vivo comparison between Cy7-1-maltotriose and Cy7-1-maltohexose in an E. coli-induced myositis murine model.

FIG. 4A illustrates in vivo fluorescence images at 18 hr post injection of either Cy7-1-maltotriose (left) or Cy7-1-maltohexose (right) showing accumulation of both probes in the right thigh muscle (E. coli). Qualitatively higher fluorescence signal in the infected muscle (right thigh) when injecting Cy7-1-maltotriose (left) compared to Cy7-1-maltohexose (right) was seen.

FIG. 4B illustrates a bar plot representation of percentage fluorescence in the infected muscle compared to whole body (left) and ratio of fluorescence in the infected thigh versus control thigh (right). Starting at 4 and 18 hr post injection, significantly higher fluorescence signal in the infected muscle (right thigh) when injecting Cy7-1-maltotriose was quantified compared to injecting Cy7-1-maltohexose (n=6, P<0.0036 and n=4, P<0.0001 respectively).

FIG. 4C illustrates a 3D rendered photoacoustic image overlaid on ultrasound image of a mouse 21 hr post injection of Cy7-1-maltotriose (top) and Cy7-1-maltohexose (bottom). Images show infected (right thigh) and control (left thigh) muscle. Evident PA signal is observed in the infected thigh muscle when injecting either compounds while minimal signal is observed in the control thigh muscle.

FIG. 4D illustrates a bar plot representation of the quantified photoacoustic signal intensity in the photoacoustic images acquired 21 hr after injection of Cy7-1-maltotriose and Cy7-1-maltohexose. The quantified signal shows significantly higher PA signal in the infected muscle compared to control muscle when injecting either compounds (left). No significant difference in PA signal was observed between the two compounds (P=0.139). A higher infected over control PA signal ratio was observed when injecting Cy7-1-maltotriose (n=6) compared to Cy7-1-maltohexose (n=3) (P<0.0004) (right). Statistical analysis was performed using one-way ANOVA. ns: no statistically significant difference (P>0.05).

FIGS. 5A-5D illustrates in vitro evaluation of Cy7-1-maltotriose in a S. aureus-infected biomaterial model.

FIG. 5A illustrates FLI and BLI images showcasing the presence and lack of S. aureus on catheters. High FLI and BLI signals were observed in catheters that were incubated with S. aureus followed by Cy7-1-maltotriose (left) compared to sterile catheters that were only incubated with Cy7-1-maltotriose (middle). Similarly, no FLI signal and only BLI signal was observed on catheters that were incubated with S. aureus only (right).

FIG. 5B illustrates total FLI and BLI signals in catheters infected with bioluminescent S. aureus and incubated with Cy7-1-maltotriose. Fluorescence quantification was plotted to the left y-axes while BLI signal plotted to the right y-axes and showed significantly higher FLI signal in infected catheters compared to sterile catheters post incubation with a solution of Cy7-1-maltotriose (*P<0.0001, n=3).

FIG. 5C illustrates axial US and PA imaging showcasing the presence and lack of S. aureus on catheters upon incubation with Cy7-1-maltotriose. An evident PA signal was observed in axial images of catheters incubated with S. aureus followed by Cy7-1-maltotriose (bottom left), compared to sterile catheters that were only incubated with Cy7-1-maltotriose (bottom right). Arrows mark the catheter's outline.

FIG. 5D illustrates a bar plot representation of the quantified photoacoustic signal intensity in the axial photoacoustic images of infected and sterile catheters post incubation with Cy7-1-maltotriose. The infected catheters had significantly higher PA signal compared to sterile catheters post incubation with Cy7-1-maltotriose (*P=0.013). Statistical analysis was performed using one-way ANOVA.

FIGS. 6A-6D illustrate in vivo evaluation of Cy7-1-maltotriose in a S. aureus wound infection murine model.

FIG. 6A illustrates BLI and FLI images of mice with a wound infected with 10⁶ CFUs of bioluminescent S. aureus 19 hr post injection of Cy7-1-maltotriose without (Untreated Group) and with (Treated Group) treatment with vancomycin for 7 days and before and after treatment. FLI (bottom-Before) shows accumulation of the probe in the S. aureus located by BLI (top-Before). In the untreated group (left panel) both BLI and FLI showcases presence of S. aureus infection in the wound after 7 days (Untreated Group-After). In the treated group (right panel), complete disappearance of S. aureus infection was observed in the FLI image and confirmed by BLI (Treated Group-After).

FIG. 6B illustrates 3D rendered PA image overlaid on US image of a mouse from Untreated (Top row) and Treated (Bottom row) groups before and after treatment. Images were acquired 20 hr post injection of Cy7-1-maltotriose. Similar to observations in FLI, the Treated group showed lower PA signal post treatment (bottom right) than that before treatment (bottom left).

FIG. 6C illustrates total in vivo fluorescence signal in wound infected with 10⁶ CFUs of bioluminescent S. aureus 19 hr after tail vein injection of Cy7-1-maltotriose. Significant reduction of FLI signal after treating the mice with vancomycin for 7 days in the treated group (n=5, P<0.0001) was observed, while no difference was observed in the untreated group before and after 7 days (n=4, P>0.99).

FIG. 6D illustrates a bar plot representation of the quantified average PA signal intensity in the PA images acquired 20 hr after injection of Cy7-1-maltotriose before and after antibiotic treatment. The quantified signal showed reduction in PA signal in the treated group after vancomycin treatment compared to before (n=5, P<0.0001). No significant difference in PA signal in the untreated group was observed in the images acquired before and after treatment (P=0.54). In addition, a higher PA signal was observed in the untreated group compared to the treated group after the treatment regimen (P<0.0001). Statistical analysis was performed using one-way ANOVA. ns: no statistically significant difference (P>0.05).

FIGS. 7A and 7B illustrate an in vivo evaluation of Cy7-1-maltotriose in a S. aureus wound infection murine model.

FIG. 7A illustrates FLI and BLI of mice with a wound infected with different amounts of bioluminescent S. aureus 22 hr post injection of Cy7-1-maltotriose. FLI images (bottom) show accumulation of the probe in the S. aureus in the same area as indicated by BLI (top).

FIG. 7B illustrates total in vivo FLI and BLI signals in wound infected with 10⁴, 10⁶ and 10⁸ CFUs of bioluminescent S. aureus (n=3, 3 and 5 respectively) and 22 hr after tail vein injection of Cy7-1-maltotriose. Fluorescence quantification was plotted to the left y-axes while BLI signal plotted to the right y-axes and showed increase in FLI signal with increase in BLI signal (r=0.928; r²=0.8612). Statistical analysis was performed using one-way ANOVA.

FIG. 8 illustrates the structure of an embodiment of a probe of the disclosure, Cy7-1-maltotriose (n=1).

FIG. 9 illustrates the ESI spectrum of compound 1a.

FIG. 10 illustrates the ¹H NMR spectrum of the compound 1a.

FIG. 11 illustrates the ¹H NMR spectrum of the compound. 1 b.

FIG. 12 illustrates the ESI spectrum of compound 2a.

FIG. 13 illustrates the ¹H NMR spectrum of the compound 2a.

FIG. 14 illustrates the ESI spectrum of compound 2b.

FIG. 15 illustrates the ESI spectrum ESI and the 1H NMR spectrum of compound 3a.

FIG. 16 illustrates an in vivo comparison between Cy7-1-maltotriose and Cy7-1-maltohexose in an E. coli-induced myositis murine model.

FIG. 16 illustrates in vivo fluorescence images at 2, 4 and 18 hr post injection of either Cy7-1-maltotriose or Cy7-1-maltohexose showing accumulation of both probes in the right thigh muscle (E. coli). Qualitatively higher fluorescence signal in the infected muscle (right thigh) when injecting Cy7-1-maltotriose (top row; n=6) compared to Cy7-1-maltohexose (bottom; n=4) is observed.

FIGS. 17A-17B illustrate in vivo evaluation of Cy7-1-maltotriose in a S. aureus-infected wound in murine model.

FIG. 17A illustrates fluorescence images of mice with a wound infected with 105 CFUs of S. aureus 24 h, 48 hr and 72 hr post tail vein injection of Cy7-1-maltotriose. Images showcase the uptake and retention of Cy7-1-maltotriose in S. aureus for up to 72 h.

FIG. 17B illustrates total in vivo fluorescence signal in wound infected with 105 CFUs of S. aureus 24 h, 48 hr and 72 hr after tail vein injection of Cy7-1-maltotriose.

FIG. 18 illustrates MALDI-TOF MS of compound 3a.

FIG. 19. Illustrates MALDI-TOF MS of compound 3b.

FIGS. 20A-20C illustrate in vitro characterization of Cy7-1-maltotriose and Cy7-1-maltohexose.

FIG. 20A illustrates murine (n=6), rats (n=4), and human (n=6) plasma and PBS (n=4) stability assessment after incubation at 37° C. for 0, 2, 4, 10 and 24 h. At the different time points samples were analyzed on analytical HPLC. Data presented as area under the peak representing the compound of interest over area of all peaks observed in the HPLC trace when monitoring at 750 nm.

FIG. 20B illustrates HPLC detection limit of both Cy7-1-maltotriose (left) and Cy7-1-maltohexose (right).

FIG. 20C illustrates absorption (solid line) and emission (dotted line) spectra of Cy7-1-maltotriose and Cy7-1-maltohexose.

FIG. 21A illustrates a HPLC traces of imaging probe Cy7-1-maltotriose after incubation in murine plasma (left), human plasma (middle), for 0 and 2 h, and after 24 hr incubation in PBS (right).

FIG. 21B illustrates a HPLC traces of imaging probe Cy7-1-maltohexose after incubation in murine plasma (left), human plasma (middle), for 0 and 2 h, and after 24 hr incubation in PBS (right).

FIGS. 22A-22D illustrate in vitro characterization of Cy7-1-maltotriose.

FIG. 22A illustrates fluorescence, ultrasound and photoacoustic images of a phantom containing different concentrations of Cy7-1-maltotriose. Evident reduction in Fluorescence and PA signal was observed with decrease in concentration.

FIG. 22B illustrates a plot showing linear correlation between Fluorescence or Photoacoustic signals and the concentration of the agent.

FIG. 22C illustrates photoacoustic imaging of a tube phantom containing 50 μM solution of Cy7-1-maltotriose at different excitation wavelengths.

FIG. 22D illustrates a bar plot representation of the quantified PA signal when exciting at different wavelengths (n=4). Statistical analysis was performed using one-way ANOVA.

FIGS. 23A and 23B illustrate in vivo validation of Cy7-1-maltotriose in an E. coli- and E. coli mutation-induced myositis murine model.

FIG. 23A illustrates (left) that FLI shows accumulation of Cy7-1-maltotriose in E. coli-infected thigh muscle at 3 and 20 hr post systemic injection (right thigh muscle). No evident accumulation of the agent in thigh muscle injected with 10⁸ CFUs of E. coli MaIG+LamB mutant (left thigh muscle). Right: Ex-vivo FLI of right and thigh muscle post excision. Image shows higher FLI signal in thigh muscle infected with E. coli compared to E. coli mutant.

FIG. 23B illustrates a bar-plot representation of quantified FLI signal in right and left thigh muscle at 3 and 20 hr post probe injection. Significantly higher fluorescence signal was found in muscle infected with E. coli (right thigh) compared to muscle infected with E. coli mutant (left thigh) at both time points (n=5, P<0.0001). Statistical analysis was performed using one-way ANOVA.

FIGS. 24A and 24B illustrate. In vivo evaluation of Cy7-1-maltotriose in a S. aureus-infected wound in murine model.

FIG. 24A illustrates FLI and BLI images of mice with wound infected with 10⁵ CFUs of S. aureus collected before and at 3, 20, 44, 72, 96, 120 and 144 hr post injection of Cy7-1-maltotriose (10 nmol, 200 μL injection). Cy7-1-maltotriose was taken up and retained in S. aureus for up to 144 h.

FIG. 24B illustrates total in vivo BLI (right y-axis) and FLI signal (left y-axis) in wound infected with 10⁵ CFUs of S. aureus when imaged before and at 20, 44, 72, 96, 120 and 144 hr post injection of Cy7-1-maltotriose (n=5). At 144 h, significantly higher FLI signal was observed compared to signal before injecting the probe (P<0.0001). The FLI image at 3 and 20 hr are shown under a different scale (shown beside the image), while the rest of the BLI and FLI images are shown under the same scale (shown on the far right). Statistical analysis was performed using one-way ANOVA.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, molecular imaging, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

In describing and claiming the disclosed subject matter, the following terminology will be used in accordance with the definitions set forth below.

The terms “administration of” and “administering” a compound or composition as used herein refers to providing a compound of the disclosure or a prodrug of a compound of the disclosure to the individual in need of treatment. The compounds of the present disclosure may be administered by oral, parenteral (e.g., intramuscular, intraperitoneal, intravenous, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “antibiotic” as used herein refers to a chemotherapeutic agent that inhibits or abolishes the growth of micro-organisms, for example, bacteria.

The phase “bacterial infection” can refer to a bacteria colonizing a tissue or organ of a subject, where the colonization causes harm to the subject. The harm can be caused directly by the bacteria and/or by toxins produced by the bacteria. Reference to bacterial infection includes also includes bacterial disease.

The term “colonization” as used herein refers to the presence of a bacterial population on the surface of such as a surgical device, the skin of a mammal but which may not be injurious to the mammal while on the skin or, if on such as a medical device surface may introduce the bacterial to a patient and thereby initiate a pathology, i.e. an infection.

The probes and compositions of the disclosure may be applied to a catheter, or a medical device that may be, for example, an endotracheal tube, a nephrostomy tube, a biliary stent, an orthopedic device, a valve, a prosthetic valve, a drainage tube, a drain, a shunt, a staple, a clip, a mesh, a film, a blood exchanging device, a port, a cardiovascular device, a defibrillator, a pacemaker lead, a wire coating, an ocular implant, an auditory implant, a cochlear implant, a dental implant, a stimulator, a drug delivery depot, a filter, a membrane, a vascular access port, a stent, an envelope, a bag, a sleeve, intravenous or other tubing, a bag, a dressing, a patch, a fiber, a pin, a vascular graft, a suture, a cardiovascular suture, or an implantable prosthesis. In some embodiments, the catheter may be a vascular catheter, a urinary catheter, an intracranial catheter, an intraspinal catheter, a peritoneal catheter, a central nervous system catheter, a cardiovascular catheter, a drainage catheter, a soaker catheter, an aspirating catheter, an intrathecal catheter, a neural catheter, a stimulating catheter, or an epidural catheter. The catheter may be a vascular catheter such as a central venous catheter, an arterial line, a pulmonary artery catheter, a peripheral venous catheter, an intravenous catheter, or an intraarterial catheter.

Bacteria that cause bacterial infections are called pathogenic bacteria. The terms “bacteria” or “bacterium” include, but are not limited to, Gram-positive and Gram-negative bacteria. Bacteria can include, but are not limited to, Abiotrophia, Achomobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothix, Pseudoanabaena, Schizothix, Spirulina, Trichodesmium, and Umezakia genera) Anaerorhabdus, Arachnia, Arcanobacterium, Arcobacter, Arthobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonela, Cedecea, Cellulomonas, Centipeda, Chlamydia, Chlamydophila, Chomobacterium, Chyseobacterium, Chyseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehlichia, Eikenella, Empedobacter, Enterobacter, Enterococcus, Erwinia, Erysipelothix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthacis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella bumetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The Gram-positive bacteria may include, but is not limited to, Gram-positive Cocci (e.g., Streptococcus, Staphylococcus, and Enterococcus). The Gram-negative bacteria may include, but is not limited to, Gram-negative rods (e.g., Bacteroidaceae, Enterobacteriaceae, Vibrionaceae, Pasteurellae and Pseudomonadaceae).

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

The term “detectably effective amount” of the labeled probe of the present disclosure is as used herein refers to an amount sufficient to yield an acceptable image using equipment that is available for clinical use. A detectably effective amount of the labeled probe of the present disclosure may be administered in more than one injection. The detectably effective amount of the labeled probe of the present disclosure can vary according to factors such as the degree of susceptibility of the individual, the age, sex, and weight of the individual, idiosyncratic responses of the individual, and the like. Detectably effective amounts of the probe of the present disclosure can also vary according to instrument and film-related factors. Optimization of such factors is well within the level of skill in the art.

The term “detectable signal” is a signal derived from non-invasive imaging techniques such as, but not limited to, fluorescence or photoacoustic detection. The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5, Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HiLyte® Fluors (AnaSpec), and DyLite® Fluors (Pierce, Inc.).

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

The term “label” or “tag” as used herein refers to a molecule that, when appended by, for example, without limitation, covalent bonding or hybridization to another moiety, for example, also without limitation, a nanoparticle provides or enhances a means of detecting the other moiety. A fluorescence or fluorescent label or tag emits detectable light at a particular wavelength when excited at a different wavelength.

The term “linker” as used herein refers to any organic structure that can form a covalent bond to a labelling moiety and also to an oligosaccharide moiety, most preferably a maltotriose, thereby attaching the label to the oligosaccharide as disclosed. The labelling moiety may, for example, be attached to the oligosaccharide of the disclosure via a linker such as, but not limited to, a 3-oxo propyl-6-oxo-hexyl chain, and the like including a plurality of oxoalkyl moieties forming a linear or substantially linear polymer. The linker may be a polyethylene glycol polymer, dextran, or the like. In alternative embodiments the linker can be a linear PEG, a multi-arm PEG, a branched PEG, and/or combinations thereof. The molecular weight of the PEG can be about 1 kDa to 100 kDa, about 1 kDa to 50 kDa, about 1 kDa to 40 kDa, about 1 kDa to 30 kDa, about 1 kDa to 20 kDa, about 1 kDa to 12 kDa, about 1 kDa to 10 kDa, and about 1 kDa to 8 kDa. The molecular weight of this PEG may advantageously be between 3000 to about 12000 kDa. Short chain (n=4 PEG) hetero- or homo-bifunctional cross-linkers may also be used.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “maltotriose” as used herein refers to a trisaccharide (three-part sugar) consisting of three glucose molecules linked with α-1,4 glycosidic bonds.

The term “maltohexose” as used herein refers to an oligosaccharide consisting of six glucose molecules linked with α-1,4 glycosidic bonds. Oligosaccharides generally contain between 3 and 9 monosaccharide. The term “detectable” refers to the ability to detect a signal over the background signal.

The term “mammal” as used herein refers to any of a class of warm-blooded higher vertebrates that nourish their young with milk secreted by mammary glands and have skin usually more or less covered with hair, and non-exclusively includes humans and non-human primates, their children, including neonates and adolescents, both male and female, livestock species, such as horses, cattle, sheep, and goats, and research and domestic species, including dogs, cats, mice, rats, guinea pigs, and rabbits.

The term “therapeutic agent” as used herein, refers to any agent, which serves to repair damage to a living organism to heal the organism, to cure a pathological condition, to combat an infection by a microorganism or a virus, to assist the body of the living mammal to return to a healthy state.

The term “therapeutic composition” as used herein, refers to an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use.

The terms “therapy,” and “therapeutic” as used herein, refer to either “treatment” or “prevention,” thus, agents that either treat damage or prevent damage are “therapeutic”.

The term “surface” as used herein refers to any surface of a surgical instrument, device, material, and the like that may be used in or contact a surgical procedure or treated subject and which may be subject to a bacterial colonization.

### Abbreviations

MRI, magnetic resonance imaging; CT computer tomography; PET, positron emission tomography; FLI, Fluorescence imaging; PAI, photoacoustic imaging; BLI, bioluminescence imaging; DBCO, dibenzoyl cyclooctyne; RT, room temperature; DCM, dichloromethane; cpm, counts per minute

### Discussion

The present disclosure provides embodiments of a labeled derivative of maltotriose for photoacoustic and fluorescent imaging of bacterial infections in an animal or human subject. The probes of the disclosure are also useful for detecting the presence of bacteria at a surgical site, or on surgical instruments or other objects and which may be transmitted to a surgical site. The label such as, but not limited to, a fluorescent dye can be attached to the anomeric carbon (reducing end) of maltotriose that has less effect on the internalization of the sugar than does functionalization at the 6″-position (non-reducing end). In embodiments of the disclosure, a variety of fluorescent dyes may be linked to the maltotriose providing that they are detectable by fluorescence detection or by the generation f a photoacoustic signal.

Embodiments of the present disclosure provide for labeled maltose-based probes, in particular labeled oligomaltose oligosaccharides, and most advantageously maltotriose, methods of making such labeled probes, pharmaceutical compositions including such labeled probes, methods of using labeled probes, methods of diagnosing, localizing, monitoring, and/or assessing bacterial infections, using labeled probes, kits for diagnosing, localizing, monitoring, and/or assessing bacterial infections, using labeled probes, and the like.

Embodiments of the present disclosure are advantageous for at least the following reasons. Maltose is used in biosynthetic and other biochemical pathways of multiple types of pathogenic bacteria (e.g., Pseudomonas aeruginosa, Escherichia coli, Bacillus subtilis, Streptococcus pneumoniae, Staphylococcus aureus, and Listeria monocytogenes). Maltose is taken up by bacteria at a rate ten times that of glucose, whereas maltose is not taken up by mammalian cells. An advantage of using labeled maltose-like probes (e.g., labeled ethyl maltoside probes and labeled maltotriose probes) is that it is a specific substrate for bacteria and can be used to image bacterial infections in mammals. Also, maltose transporters are present in most pathogenic bacteria, so labeled probes can be used to image multiple types of infections and/or differentiate between bacterial and viral infections. Ethyl maltoside and maltotriose are pseudo-oligosaccharides, which are transported but not metabolized by the maltose-maltodextrin system of E. coli.

In an embodiment of the disclosure, the labeled probe can be used to image bacterial infections of Gram-positive and Gram-negative bacteria including, but not limited to, E. coli, S. aureus, and Ps. aeruginosa. In particular, the present disclosure includes methods relating to non-invasive imaging using labeled maltoside probes as herein disclosed.

Embodiments of the present disclosure further include methods for imaging a sample (e.g., tissue or cell(s)) or a subject (e.g., mammal), that include the steps of contacting a sample with or administering to a subject a labeled probe (i.e., a fluorescent maltotriose probe of the disclosure) and imaging with a fluorescent or photoacoustic imaging system. The imaging can be performed in vivo and/or in vitro. In particular, embodiments of the present disclosure can be used to image bacterial infection. In this regard, the sample or subject can be tested to determine if the sample or subject has a bacterial infection, monitor the progression (or regression) of the bacterial infection, assess the response of the bacterial infection to treatment, and the like. In an embodiment, the tissue or cells can be within a subject or have been removed or isolated from a subject.

As noted above, these labeled probes can be associated and/or correlated with a bacterial infection, thus the detection of the probe in a location can be used to identify the location of the bacterial infection. Additional details regarding the labeled maltoside probe are described herein.

In each synthesis of the probes of the disclosure, it should be noted that alternative protecting groups can be used to replace the acetyl group, the trityl group, and/or nosylate group so as long as any replacement(s) permit the synthesis to produce the desired labeled probe. For example, the acetyl group can be replaced with one of the following: benzoyl, benzyl, methoxymethyl, allyl, t-butyldimethylsilyl, tetrahydropyranyl, t-butyldiphenylsilyl and t-butyl; the trityl group can be replaced with one of the following: methoxyphenyldiphenylmethyl, t-butyldimethylsilyl, tetrahydropyranyl, t-butyldiphenylsilyl and t-butyl; and the nosylate group can be replaced with one of the following: tosylate, triflate, brosylate, mesylate, and thiolate groups.

Discussions focusing on the labeled ethyl maltoside probes and labeled maltotriose probes are not limiting to the scope of the disclosure, rather those discussions are merely describing an exemplary embodiment of the present disclosure.

### Dosage Forms

Embodiments of the present disclosure can be included in one or more of the dosage forms mentioned herein. Unit dosage forms of the pharmaceutical compositions (the “composition” includes at least the labeled probe of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the labeled probe, pharmaceutically acceptable salts thereof, with other chemical components, such as physiologically acceptable carriers and excipients. One purpose of a pharmaceutical composition is to facilitate administration of labeled probe to a subject (e.g., human).

Solvates of the compounds of the disclosure are also contemplated herein. Solvates of the compounds are advantageously hydrates.

The amounts and a specific type of active ingredient (e.g., a dye conjugated maltotriose probe) in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

Accordingly, provided is the development and preclinical evaluation of novel fluorescent derivatives of maltotriose against a number of bacteria strains and murine infection models. Maltotriose-based probes have the unique ability to be specifically internalize into both Gram-positive and Gram-negative bacteria mediated by a maltodextrin transporter that is not present in mammalian cells. Synthesized fluorescent derivatives of maltotriose can be functionalized with a Cy7 dye at the anomeric carbon in high yields using copper-free click chemistry.

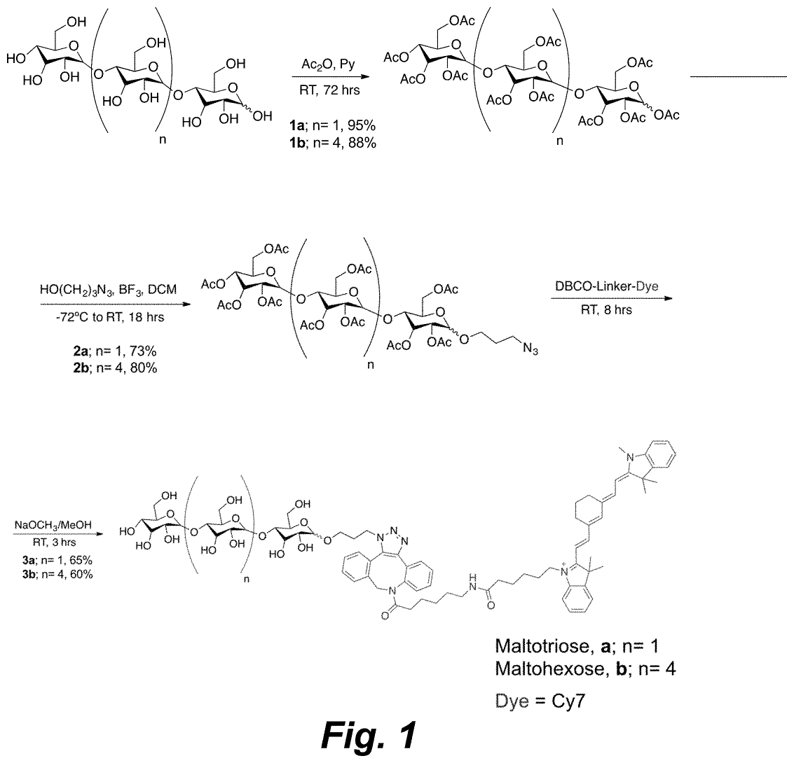

The present disclosure provides a modified synthetic route that produces an azide-1-functionalized maltotriose or maltohexose using an adapted glycosylation procedure ((Khamsi et al., Carbohydrate Res. 357: 147-150 (2012)) in two high yielding steps. Such an intermediate simplifies the functionalization of the maltotriose and maltohexose scaffold to a variety of signaling agents using copper-free click chemistry or copper catalyzed azide-alkyne reaction. Cy7 dye was initially chosen due to its great photo and chemical stability and linked to maltotriose (n=1) or maltohexose (n=4) through azide-DBCO copper-free click chemistry (FIG. 1).

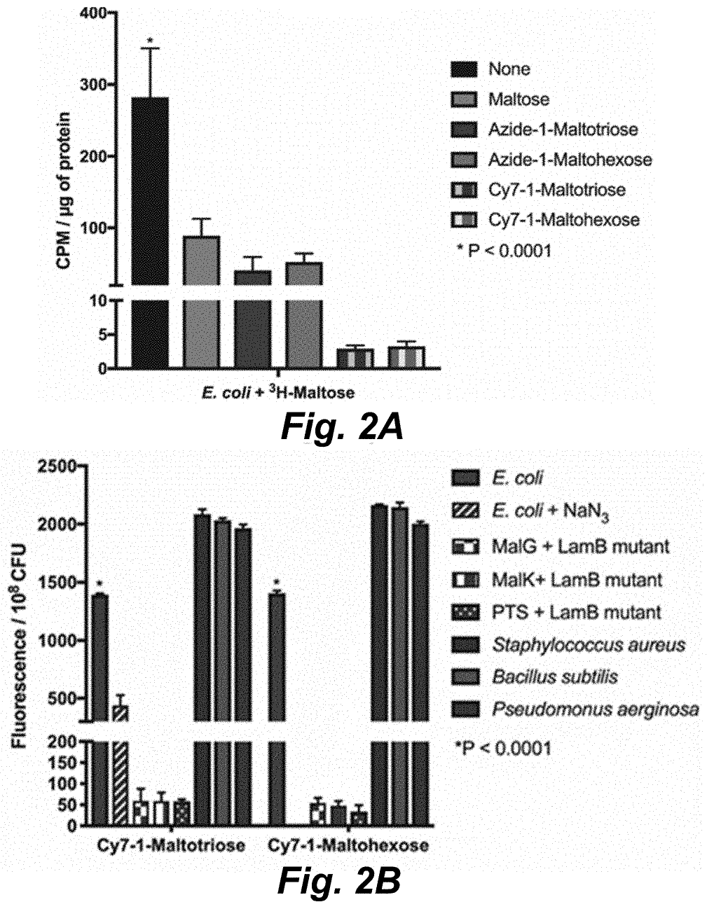

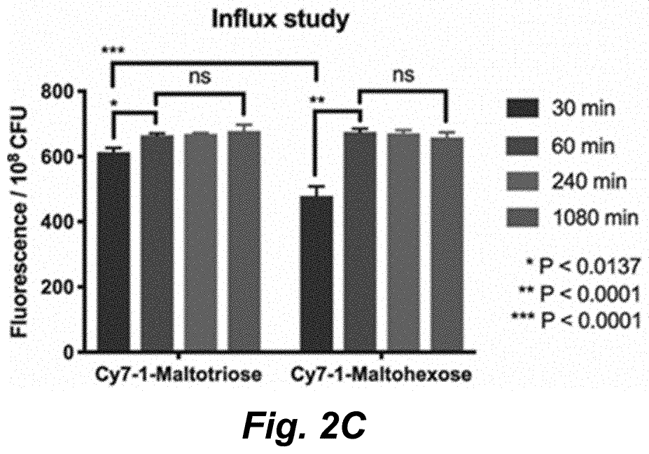

Following synthesis of the probes, the effects of the azide and Cy7 motifs on the ability of maltotriose and maltohexose to internalize into bacteria were assessed by running a competition assay between the tested derivatives and ³H-maltose as well as uptake studies (FIGS. 2A and 2B, respectively). Both probes were taken up in a wide variety of gram-positive and gram-negative bacterial strains (FIG. 2B). The specificity of the probes for imaging live bacteria that contain the ABC transporter was demonstrated in uptake studies with azide-inactivated E. coli and E. coli-mutants that lack components of the ABC transporter (P<0.0001, n=3) (FIG. 2B).

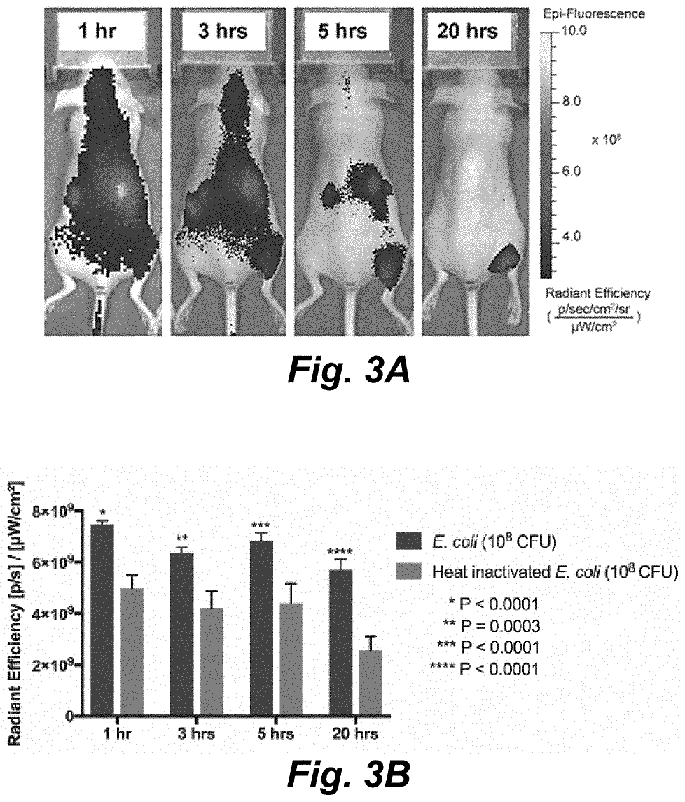

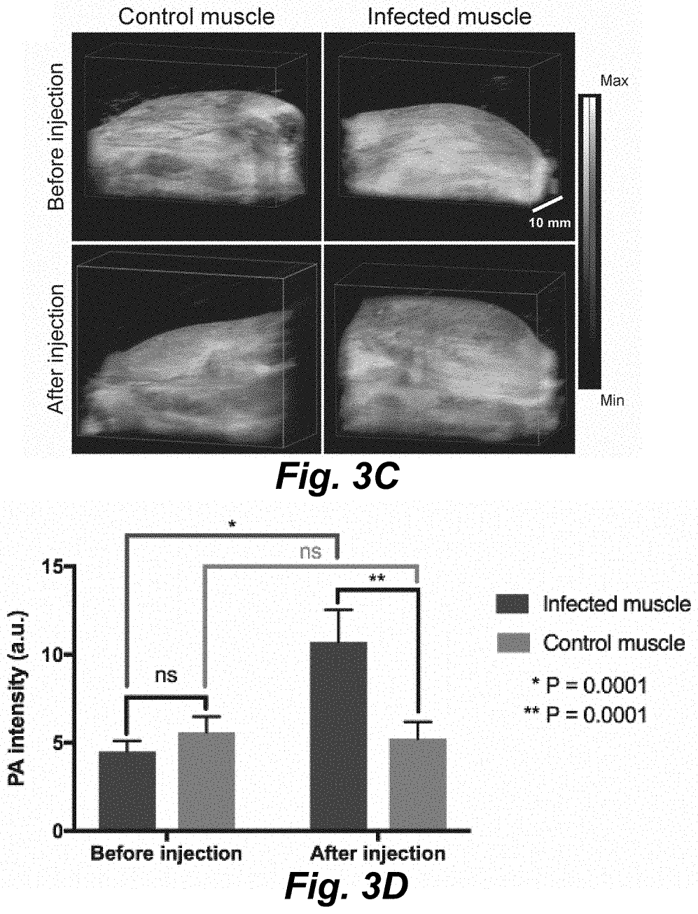

Preclinical evaluation of Cy7-1-maltotriose in E. coli-induced myositis murine model using FLI and PAI was conducted. Fluorescence imaging was used as the primary tool to assess the specificity and uptake kinetics of the probe in bacterial infections because it allows whole-mouse imaging and provides information that can be compared to previous optical imaging agents for infection. The kinetics of accumulation of the probe were examined and illustrate rapid clearance of Cy7-1-maltotriose through the kidneys. Notably, specific signal accumulation at the site of the infected muscle occurred very rapidly (as early as one hour) (FIGS. 3A and 3B). In addition, photoacoustic images were also able to show significant differences between the infected and control muscle (FIGS. 3C and 3D). PAI is advantageous due to the enhanced imaging penetration depth.

The optimum maltodextrin scaffold to utilize in our bacterial infection imaging agent was developed assessing the difference in internalization kinetics, stability and retention between maltodextrins in a variety of bacterial strains (Axer et al. ChemMedChem 13, 241-250 (2018); Dippel & Boos J. Bacteriol. 187: 8322-8331 (2005); Dutzler et al., Structure 4: 127-134 (1996); Quiocho et al., Structure 5: 997-1015 (1997); Oldham et al., Proc. Nat. Acad. Sci. U.S.A. 110: 18132-18137 (2013); Sauvageot et al. J. Bacteriol. 199: e00878-16 (2017); Licht et al., Res. Microbiol. 170: 1-12 (2019); Dumont et al. Life Science Alliance 2: e201800242 (2019)). A comparison between the maltotriose and maltohexose analogues of the imaging agent showed specificity to bacterial infections in rat models (Ning et al., Nat. Mater. 10: 602-607 (2011); Takemiya et al., JACC Cardiovasc. Imaging 12; 875-886 (2019)). In vitro competitive and uptake studies looked similar to that of Cy7-1-maltotriose (FIGS. 2A and 2B, respectively).

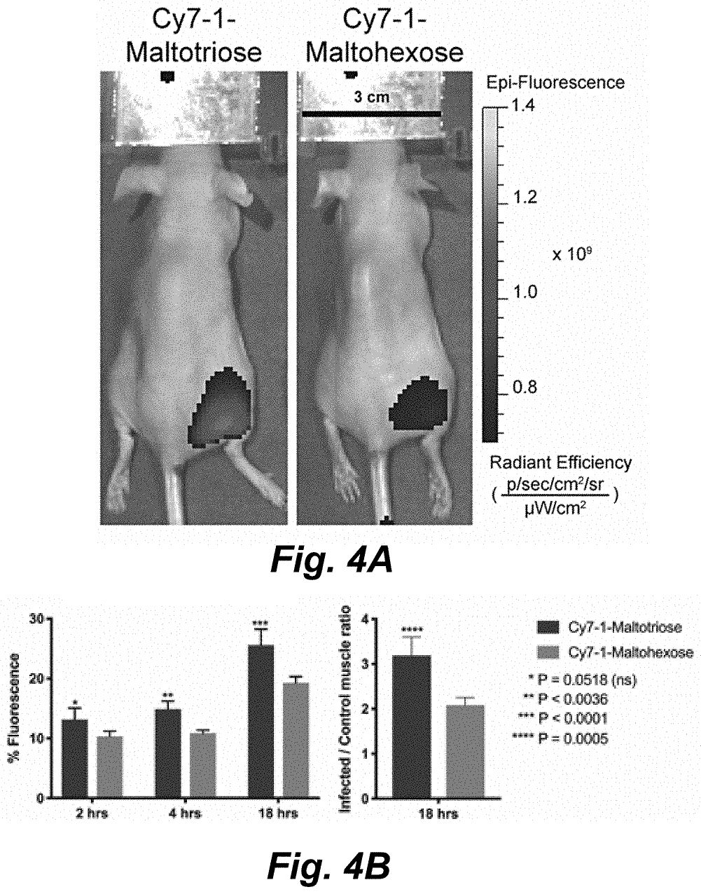

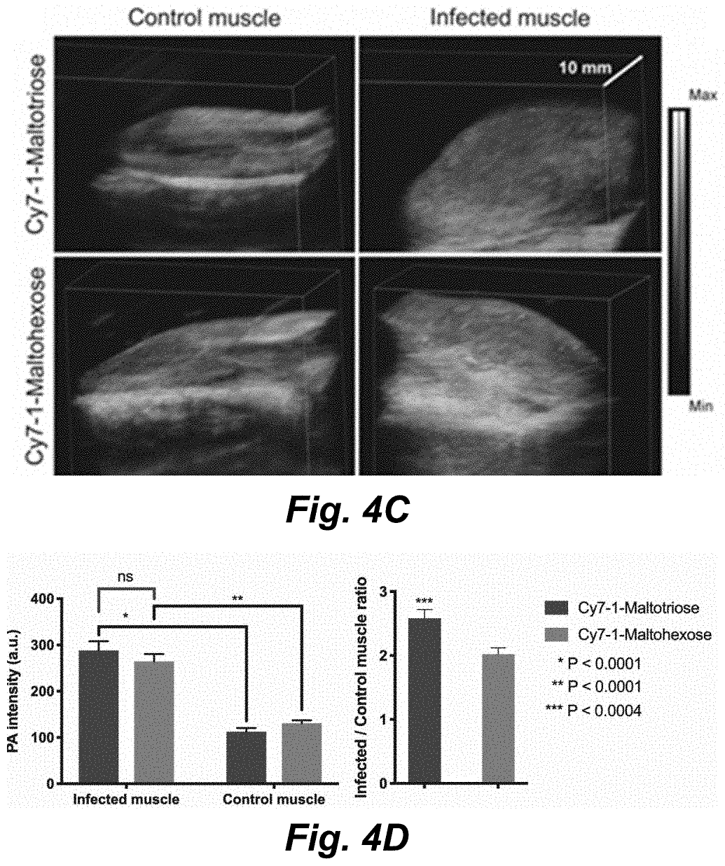

A comparison study was then conducted in the E. coli-induced myositis murine model and demonstrated the pharmacokinetic advantages of maltotriose over maltohexose in vivo. As shown in FIGS. 4A-4D, both Cy7-1-maltotriose and Cy7-1-maltohexose showed specific uptake in the infected muscle (right thigh) which is distinguishable from the control muscle (left thigh) (FIGS. 4A and 4B). 18 hrs post-injection, Cy7-1-maltotriose had 2.6 fold higher fluorescence compared to that of Cy7-1-maltohexose (15.5±5.0×10⁹ and 6.02±0.5×10⁹ radiance efficiency respectively; P<0.0001, n=6 and 4 respectively). This can be attributed to the faster clearance of the Cy7-1-maltohexose as compared to Cy7-1-maltotriose from circulation due to higher hydrophilicity (C Log P=−5.8 vs. −12.3 for maltotriose and maltohexose, respectively). The in vivo studies matched the observations made in in vitro influx studies where a much faster uptake was observed for the maltotriose derivative (FIG. 2C).

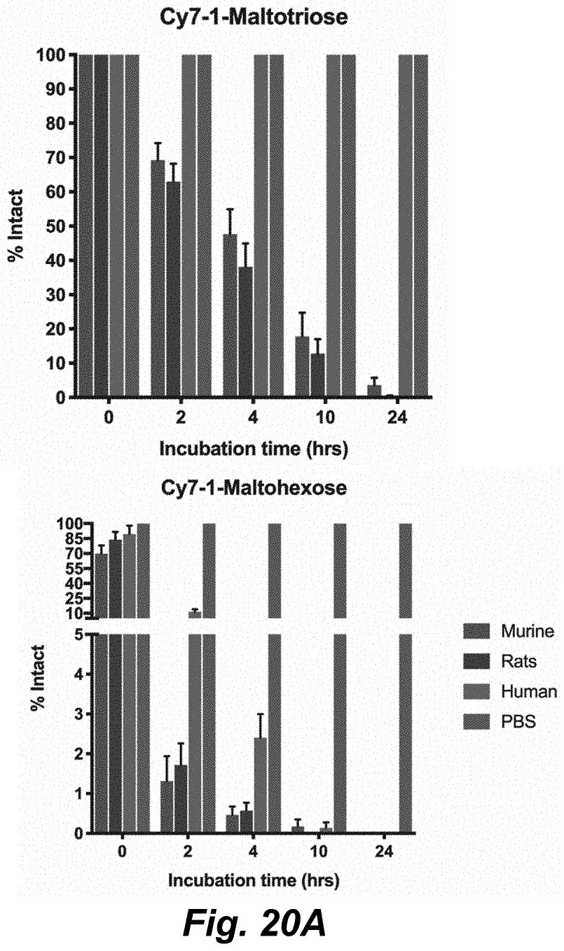

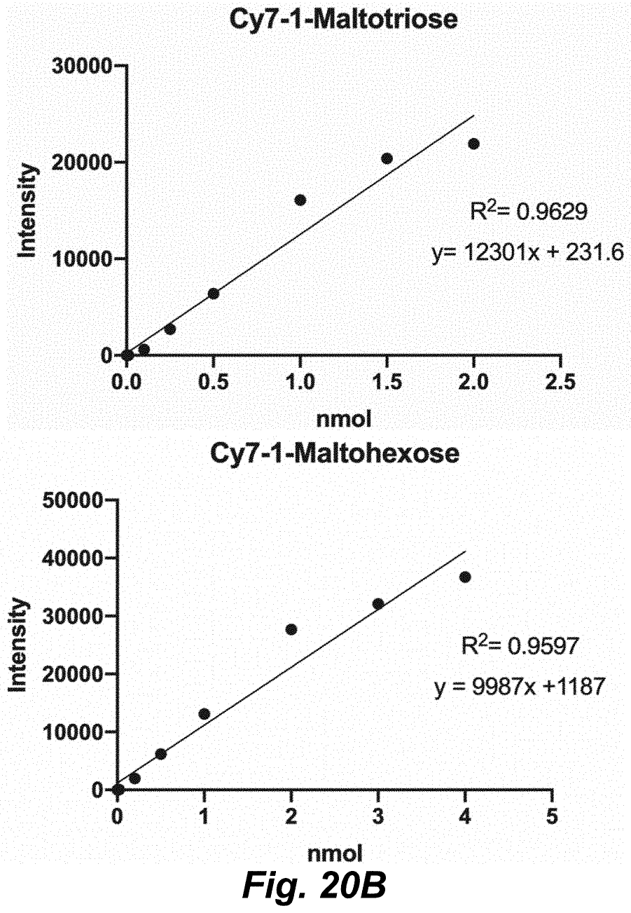

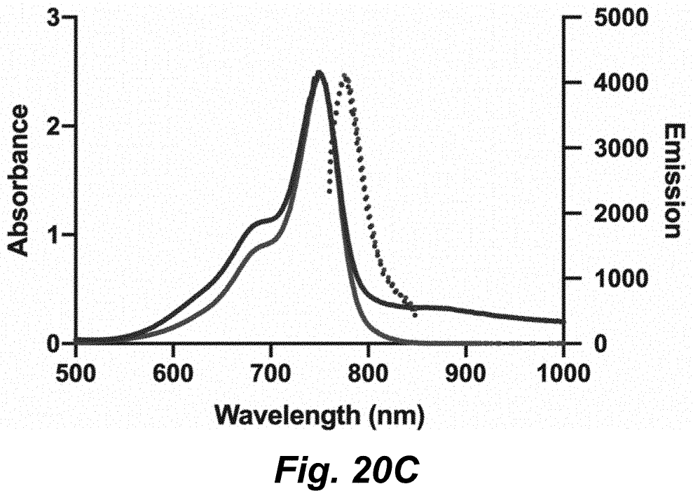

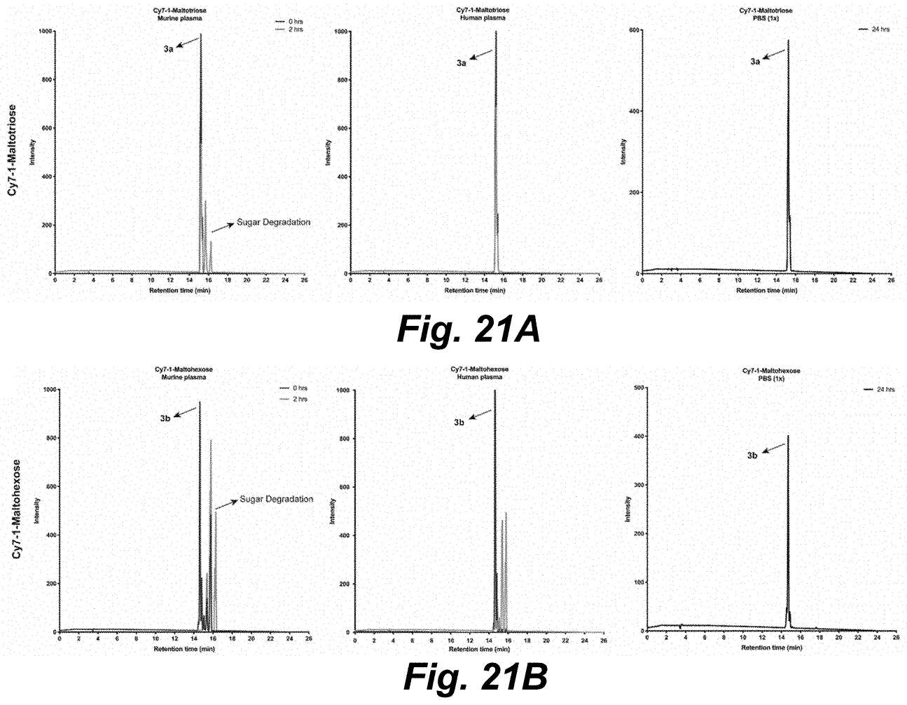

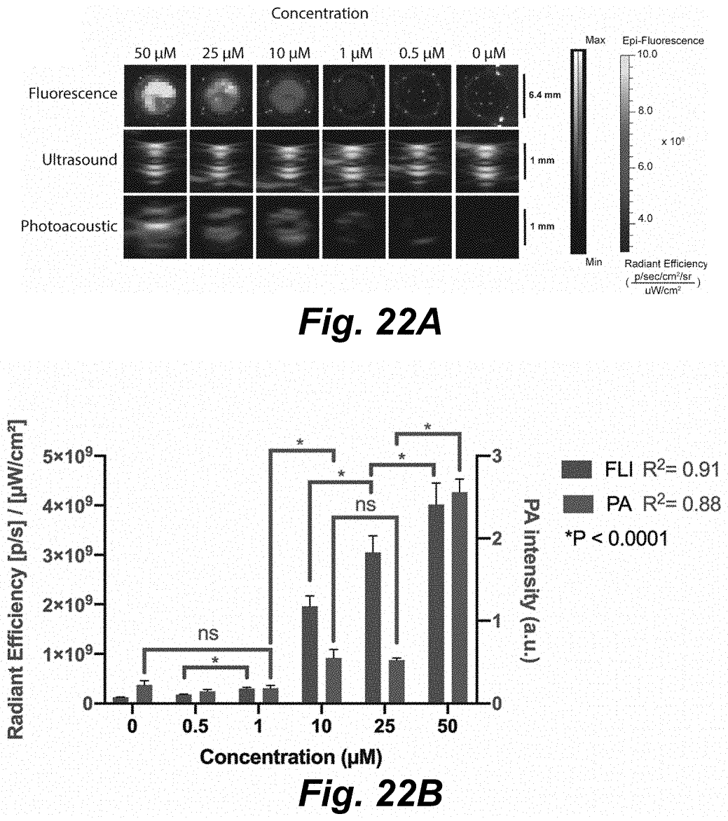

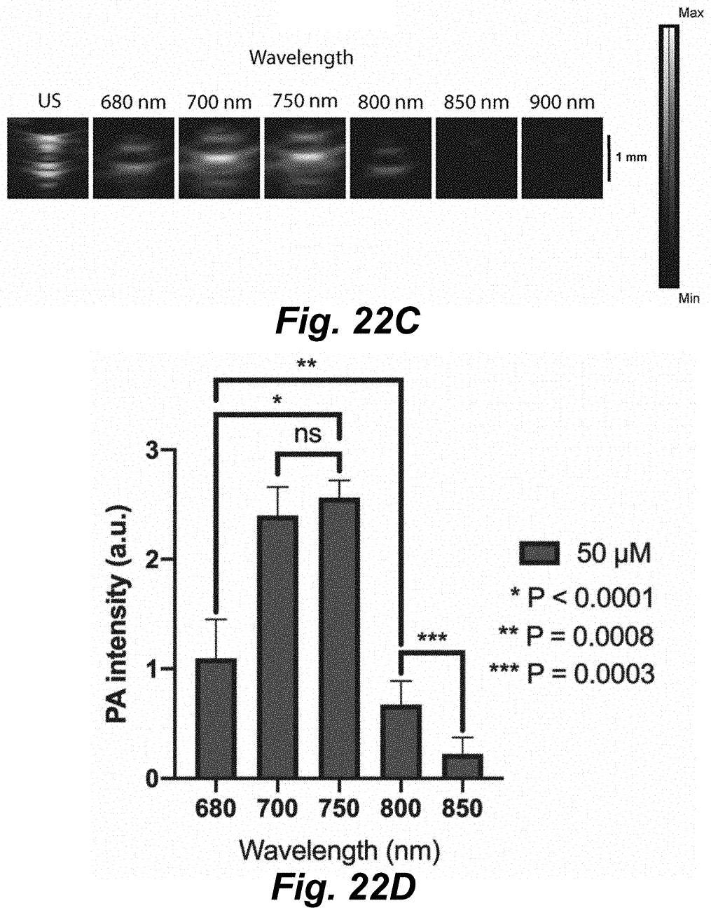

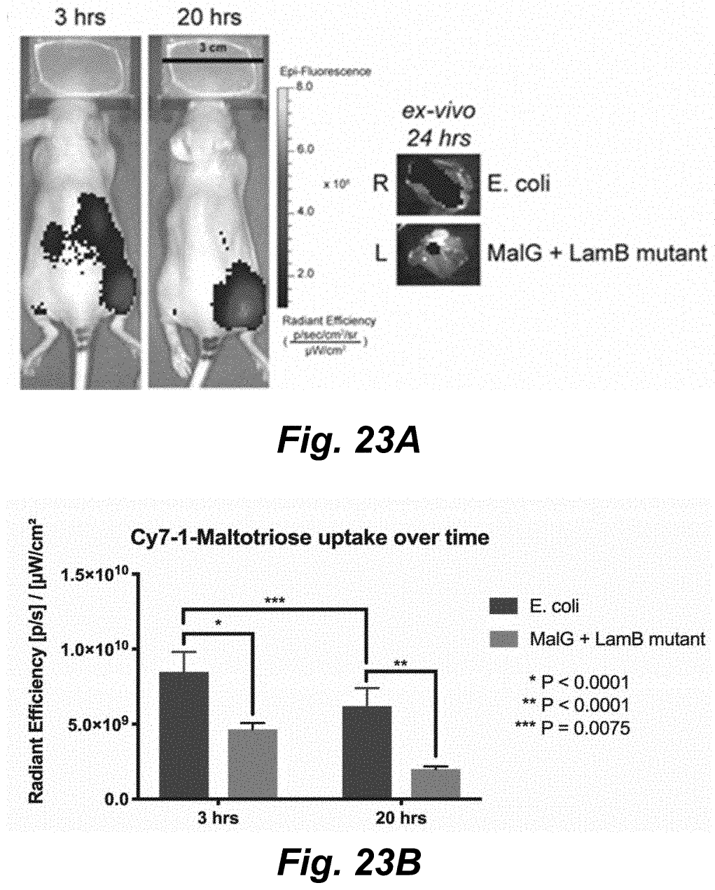

Stability studies on both compounds in murine, rat and human plasma as well as PBS overtime, showed substantial differences in stability between the maltotriose and maltohexose derivatives. Specifically, Cy7-1-maltohexose is shown to rapidly break down into what we hypothesize to be smaller sugar forms in mere minutes in plasma (less than 2% intact by 2 hrs in rat and murine and around 10% in human, FIGS. 20A (right) and 21B). While around 70% of Cy7-1-maltotriose was intact in murine and rat after 2 hr and no degradation of maltotriose in human plasma was observed (FIGS. 20A (left) and 21A). In addition, both maltotriose and maltohexose were stable in PBS for up to 24 hr (FIGS. 20A and 21 (right)). In a PA imaging study, slightly higher PA intensity in the infected thigh using Cy7-1-maltotriose (n=6) was observed compared to Cy7-1-maltohexose (n=3) (288.2 vs 264.6 a.u. respectively) yet this difference was not significant (P=0.139). But when looking at the PA intensity ratio of infected over control muscle, significantly higher ratio using the maltotriose derivative versus maltohexose was observed (2.5 vs 2 respectively). This further resembles the data shown in the FLI study and is most likely due to the lower sensitivity of PA in detecting Cy7 which is more geared for fluorescence imaging rather than photoacoustic detection (FIG. 22B).

The slower bacterial-uptake of the maltohexose derivative, its faster clearance due to its hydrophilicity and its lower plasma stability demonstrates that Cy7-1-maltotriose is advantageous as a bacterial-imaging probe.

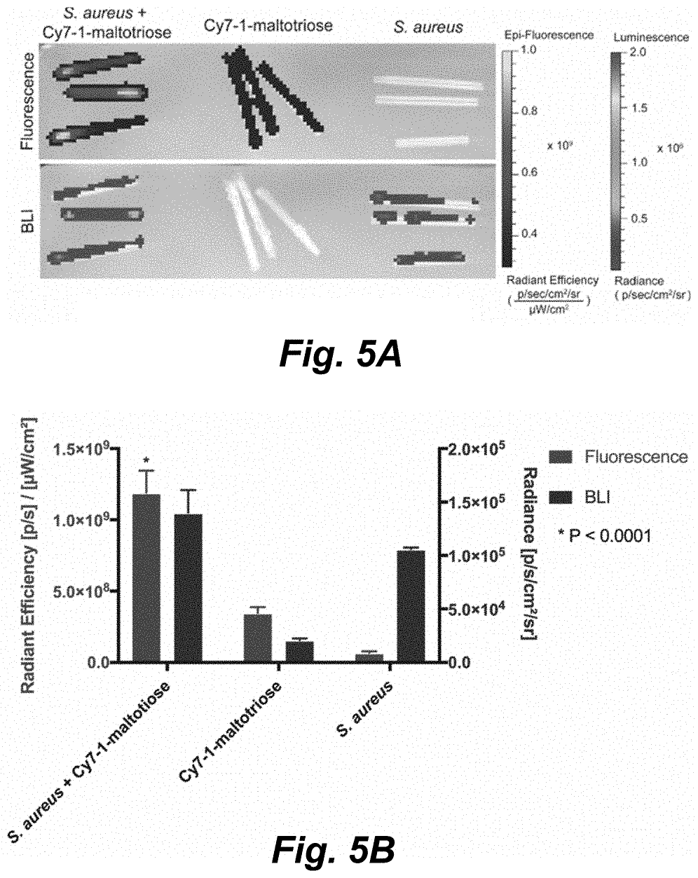

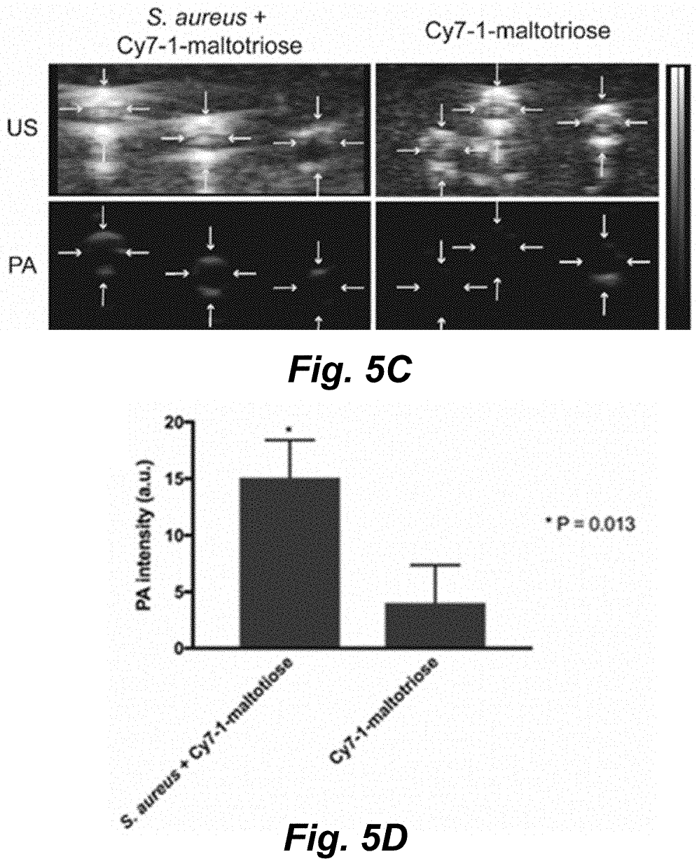

The Cy7-1-maltotriose probes of the disclosure are advantageous for the detection and monitoring of treatment of bacterial infections in susceptible sites (i.e. wounds, surgical sites and medical implants). Device-associated infections account for around 25.6% of all healthcare associated infections in the United States (Arciola et al., Nat. Revs. Microbiol. 16: 397-409 (2018)). Current periprosthetic joint infection (PJI) diagnostic tools necessitate sample collection from a prosthetic site and are divided to culture-based tools (ex. peri-implant tissue culture, synovial culture and histology) (Fernández-Sampedro et al. BMC Infect. Dis. 17: 592 (2017)) and culture-independent tools (ex. Ibis PLEX-ID technology (Arciola et al., Int. J. Artif. Organs 34: 727-736 (2011)), MALDI-TOF mass spectroscopy (Harris et al. Int. J. Artif. Organs 33: 568-574 (2010)), next-generation sequencing (Deurenberg et al. J. Biotechnol. 243: 16-24 (2017)). Thus, a non-invasive tool for PJI diagnostic which does not rely on sampling from the site will be useful and allow differentiation from inflammation which is often confused with infections in these situations as they present with similar symptoms. Since S. aureus is the most prevalent pathogen in medical device infections and accounts for about 32% of medical device infections (Arciola et al., Nat. Revs. Microbiol. 16: 397-409 (2018)), S. aureus infections of biomaterials were assessed using PAI and FLI upon incubation with Cy7-1-maltotriose. As shown in FIGS. 5A-5D, Cy 7-maltotriose is able to differentiate infected from uninfected catheters both using FLI and PAI and provides the possibility of using PAI to image implants particularly in the joints, fracture fixtures and screening for infections.

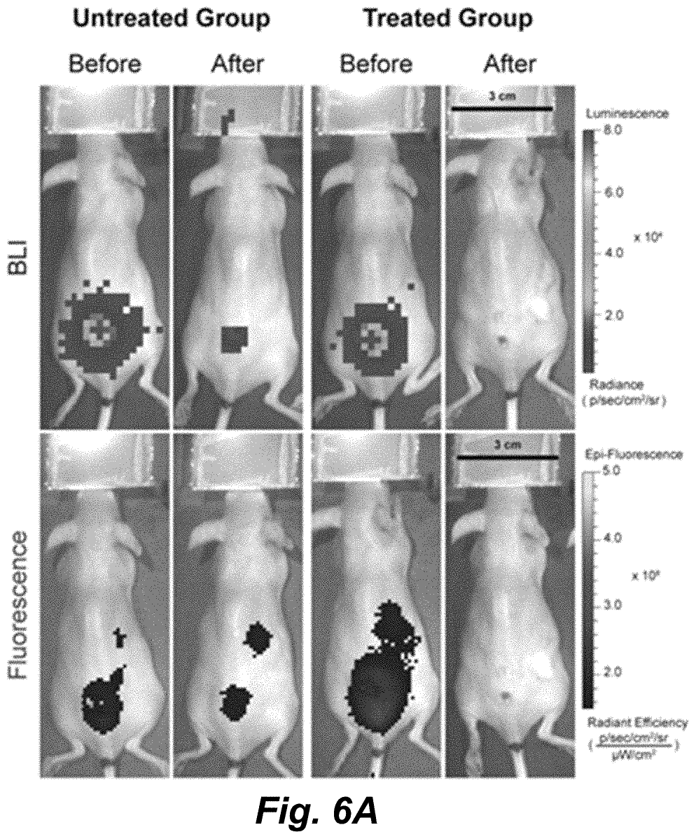

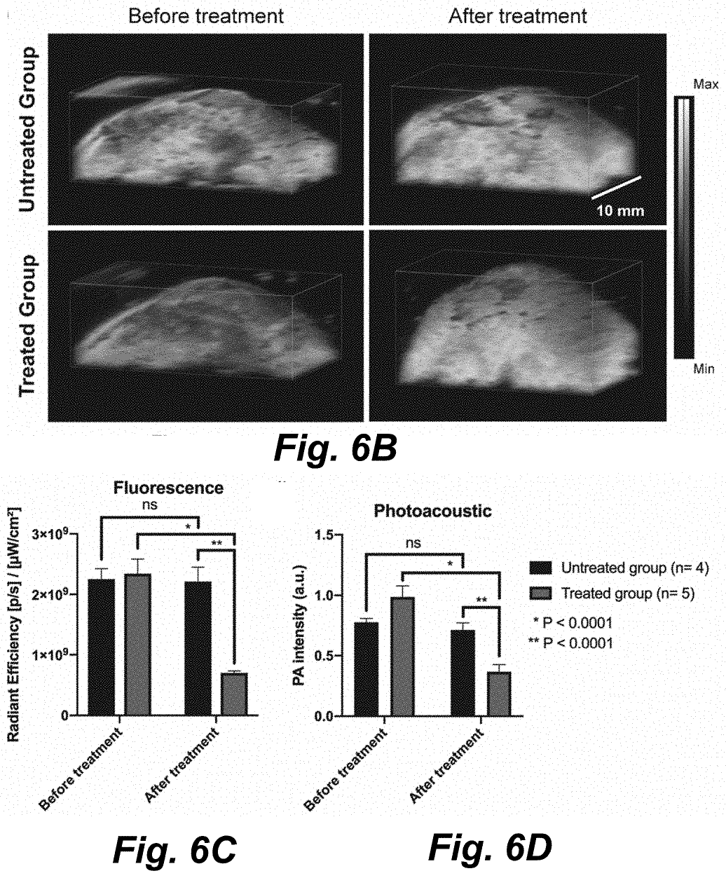

The ability of the probes of the disclosure to assess wound infections as well as determining the effectiveness of antibiotic treatment in vivo. Antibiotic treatment reduced the bacterial burden and the probe uptake reflected this decrease and showed significant differences in signal between treated and untreated groups in both FLI and PAI (FIGS. 6A-6D). In addition, both FLI and PAI were able to distinguish between the treated and untreated groups (FIGS. 6C and 6D).

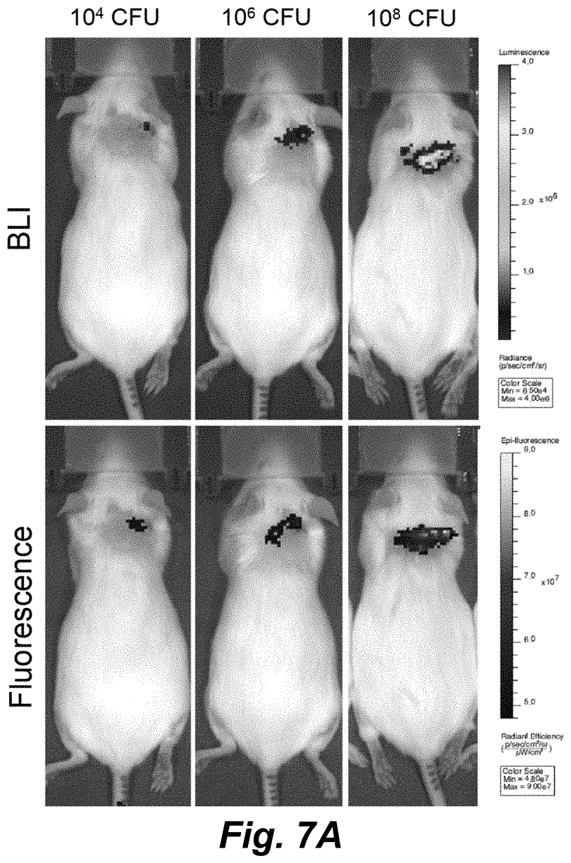

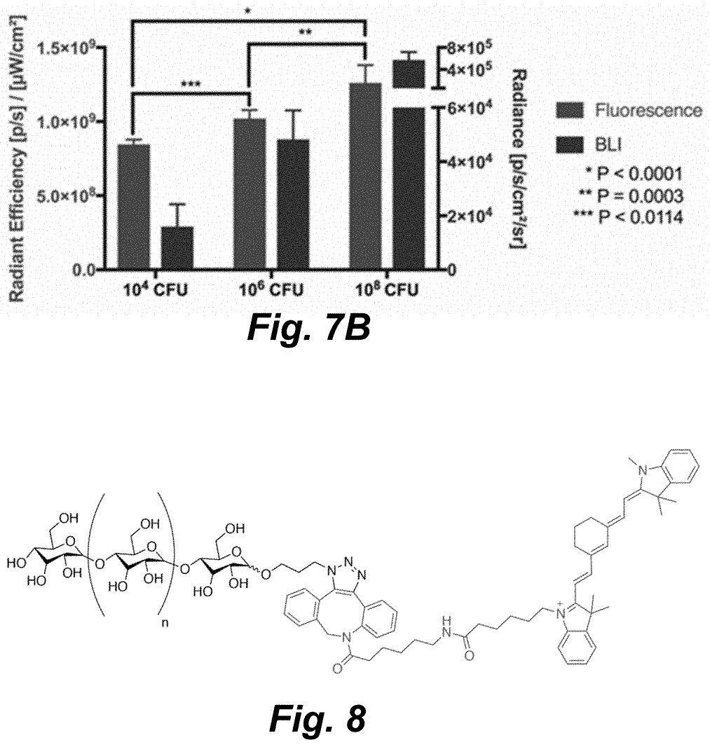

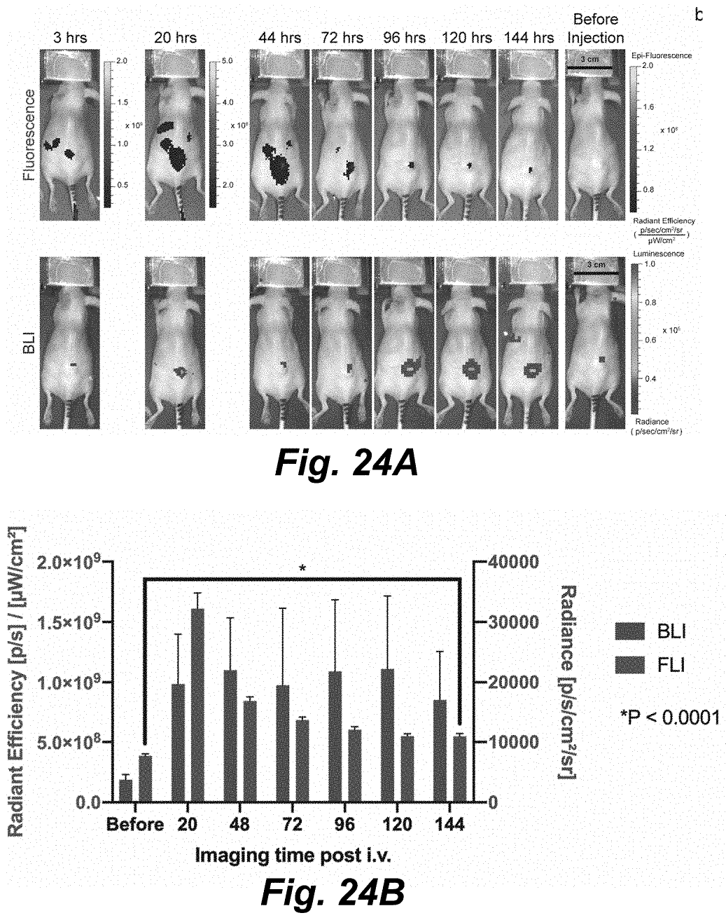

FLI evaluation showed an increase in FLI signal (i.e. Cy7-1-maltotriose accumulation) with increase in CFU of S. aureus in the wound (i.e. increase in BLI signal). Wounds infected with as low as 10⁴ CFU were detectable by FLI of Cy7-1-maltotriose and showed strong correlation with the location of the BLI signal (FIGS. 7A and 7B). In addition, imaging done with the S. aureus wound model showed that once injected, the probe remains at the infected wound for up to 144 hr post injection, which allows serial imaging without having to administer the probe repeatedly (FIGS. 24A and 24B).

### Synthesis of the Fluorescent Probes

Initially, synthesizing the fluorescent maltotriose probe, and the maltohexose derivative for comparison followed a previously reported procedure (Ning et al., Nat. Mater. 10: 602-607 (2011)). The reported probes were prepared by synthesizing azide-1-maltohexose in four steps which was then functionalized to a fluorescent dye though copper-assisted click chemistry (Ning et al., Nat. Mater. 10: 602-607 (2011)). However, it was found that the preparation of the azide-1-derivative was low yielding especially when using maltotriose as starting material (final step yield approximately 10% and 20% for maltotriose and maltohexose, respectively). Accordingly, a modified synthetic route was established that produced the same azide-1-functionalized maltotriose (and maltohexose) using Fischer glycosylation in two high yielding steps instead of four steps (Khamsi et al., Carbohydrate Res. 357: 147-150 (2012)). Cy7 dye was then linked to maltotriose (or maltohexose) though azide-DBCO copper-free click chemistry.

Azide-functionalized intermediates at the anomeric carbon of maltotriose and maltohexose (compound 2a and 2b respectively) were synthesized using copper-free click chemistry to allow ease of functionalization with a variety of signaling agents. Compound 1a was synthesized using an adapted procedure used to prepare the previously published maltohexose derivative (compound 1 b) (Ning et al., Nat. Mater. 10: 602-607 (2011)).

Maltotriose (0.51 mmol, 1 eq) was completely dissolved in pyridine (10 mL) under inert gas before addition of acetic anhydride (5 mL) and mixing at room temperature for 72 h. After solvent evaporation under vacuum, the crude mixture was dissolved in ethyl acetate and a workup in sodium carbonate (1M), hydrochloric acid (0.1M) and brine was conducted. After collecting and drying the organic layer, the off-white precipitate was dissolved in dichloromethane and purified by flash column chromatography to afford compound 1a as a white precipitate in 94% yield.

Compound 2a was produced from 1a using an adapted procedure (Khamsi et al., Carbohydrate Res. 357: 147-150 (2012)). Briefly, Compound 1a was placed in a flask in a dry ice in acetone bath (−78° C.) under dry conditions before adding 3-azido-1-propanol. After 15 min, boron trifluoride was added, and reaction stirred for 2 hr on dry ice before stirring overnight at room temperature. After quenching the reaction with triethylamine (TEA), the solvent was removed under vacuum. The resulting precipitate was then dissolved in ethyl acetate, washed with brine and purified by flash chromatography resulting in a mixture of compound 2a as well as partially deacetylated 2a (FIG. 1) as previously reported (Khamsi et al., Carbohydrate Res. 357: 147-150 (2012)). Since the glycosylation was successful and the final product was going to be fully deacetylated, the reaction was carried on the mixture.

The mixture was functionalized with a commercially available fluorescent dye coupled to dibenzoyl cyclooctyne (Cy7-DBCO), through strain-promoted azide-alkyne [3+2] cycloaddition reaction, after dissolving in a dichloromethane:methanol (1:1) solution and stirring at room temperature overnight. Alternative fluorescent dyes known to those in the art may also be employed. Sodium methoxide was then added to the crude mixture, to deprotect the acetate groups, and left stirring at room temperature for 3 hr before quenching with acetic acid. The solvent was removed under vacuum and the crude mixture dissolved in methanol and purified by reverse phase HPLC to afford compound 3a in 65% yield. In addition, the Cy7-derivative of maltohexose was also prepared following the same synthetic route producing compound 3b in 60% overall yield (FIG. 1).

### In Vitro Evaluation

A competition binding assay between the synthesized derivatives and ³H-maltose that is taken up in bacteria by the ABC transporter, was conducted. A significant reduction in ³H-maltose uptake in E. coli was observed when pre-incubated with maltose, maltotriose, maltohexose and azide, or Cy7 derivatives of maltotriose and maltohexose (FIG. 2A). Adding the azide or Cy7 functional groups on the anomeric carbon of maltotriose or maltohexose did not show any effect on their ability to block the uptake of ³H-maltose.

A direct assessment of the ability of Cy7-1-maltotriose 3a and Cy7-1-maltohexose 3b to be specifically taken up by a variety of bacteria strains containing the ABC transporter was also conducted. FIG. 2B illustrates the ability of this probe to be taken up by E. coli, Staphylococcus aureus, Bacillus subtilis and Pseudomonas aeruginosa. Control studies, where azide-inactivated E. coli or E. coli strains lacking components of the maltodextrin transporter were also evaluated and showed minimal uptake (FIG. 2B).

In Vivo Evaluation in E. coli-Induced Myositis Murine Model

Compound 3a was evaluated in vivo in E. coli-induced myositis and S. aureus wound-infection murine models. Fluorescence images of the mice gathered over time illustrated rapid accumulation of compound 3a in the mouse right thigh that had been infected with E. coli. Accumulation was not observed in the left thigh of the same animal that had been injected with heat-inactivated E. coli (FIG. 3A). In addition, significantly higher signal intensity in the right thigh compared to the left thigh starting at the one-hour imaging time point was observed and the signal difference increased over time (FIG. 3B).

The same animal model was then used to monitor the infection site using photoacoustic imaging. As a control, photoacoustic images of both infected and control thigh muscle were collected before and after probe injection. Qualitatively, higher photoacoustic signal in the infected thigh was observed when imaged after probe injection compared to that of control muscle (FIG. 3C, bottom). In addition, when quantifying the photoacoustic signal, a significantly higher photoacoustic signal was found in the post-probe injection images of the infected thigh muscle compared to that before injection or to control thigh muscle (P=0.0006 and 0.0059 respectively) (FIG. 3D). No significant difference in photoacoustic signaling between images of control and infected thigh muscle was observed before injecting the probe (P=0.70). Similarly, no significant difference in photoacoustic intensity in control muscle was found between images collected before and after probe injection (P=1.00) (FIG. 3D).

In the same animal model, a comparison study between the maltotriose and maltohexose derivatives (compound 3A and 3B, respectively) was conducted. The same amount of probe (5 nmol) was injected via the tail vein and mice were imaged at 2, 4 and 18 hr post injection. Higher fluorescence signal in the infected thigh (right) was observed in the mice injected with compound 3a (FIG. 3E, left). In addition, fluorescence signal in the infected muscle was quantified and normalized to either whole body (% fluorescence) (FIG. 4B left) or to control muscle (ratio) (FIG. 4B right) and showed approximately 1.5 times higher fluorescence signal in the infected thigh of mice injected with the maltotriose derivative (compound 3a) compared to those injected with the maltohexose derivative (compound 3b).

In photoacoustic imaging, significantly higher PA intensity in the infected thigh compared to control thigh was observed using either probes (P<0.0001) (FIGS. 4C and 4D). Higher PA intensity in the infected thigh of mice injected with Cy7-1-maltotriose (n=6) was observed compared to ones injected with Cy7-1-maltohexose (n=3), but this difference was not significant (FIG. 4D left, P=0.1390). However, significantly higher infected over control muscle PA signal ratio was observed when injecting Cy7-1-maltotriose compared to injecting Cy7-1-maltohexose (FIG. 4D right, P<0.0004).

To further assess the specificity of Cy7-1-maltotriose to bacteria containing the maltodextrin transporter, a similar study where a MaIG+LamB mutant of E. coli was injected instead in the left thigh. In vivo fluorescence imaging at 3 and 20 hr post injection also showed rapid and significantly higher accumulation of Cy7-1-maltotriose in the E. coli infected thigh (right thigh muscle) compared to the left thigh infected with E. coli mutation (n=5, Figure S8, P<0.0001).

In Vivo Evaluation in S. aureus-Infected Wound Murine Model

10⁶ CFU of S. aureus were inoculated into the wound one day before injecting Cy7-1-maltotriose via the tail vein. This bacterial strain (Xen 36) is kanamycin resistant and is bioluminescent, allowing its detection through BLI. BLI and FLI images were collected 19 hr post injection of the probe while PA imaging was conducted 20 hr post injection. After confirmation of the presence of bacteria through BLI imaging, FLI and PAI was conducted (FIG. 6, before treatment). Mice were then divided into two groups where one was administered subcutaneously a therapeutic dose of vancomycin twice daily (Treated group, n=5) while the other group was not treated with vancomycin (untreated group, n=4). After seven days of antibiotic treatment, Cy7-1-maltotriose was administered and imaging completed 20 hr post injection. No BLI nor FLI signal and minimal PAI signal in the wound were observed in the treated group post-treatment (FIGS. 6A and 6B, Treated group-After). The untreated group showed evident BLI, FLI and PAI signal in the wound. In addition, a significant decrease in the fluorescence and photoacoustic signal (4.33±0.96×10⁸ vs 1.48±0.15×10⁸ radiance efficiency and 0.99±0.09 vs 0.37±0.06 a.u. respectively, P<0.0001, n=4) was observed in the images collected post treatment compared to before treatment (FIGS. 6C and 6D).

In a similar animal model, different amounts of the bioluminescent Xen 36 strain Staphylococcus aureus (10⁴, 10⁶ or 10⁸ CFU; n=3, 3, and 5, respectively) were inoculated subcutaneously in a small wound formed on the back of the mice. FLI images 18 hr post injection showed accumulation of Cy7-1-maltotriose in the wound in all three mice groups where the location of the FLI signal directly correlated to that of the BLI signal (FIG. 7A). In addition, significant increase in the quantified fluorescence signal was observed with increase in quantified BLI signal (i.e. increase in CFU in the wound) (FIG. 7B).

### In Vitro Imaging of Biomaterial Infections

Sterilized catheters were incubated in a solution of 10⁶ CFU of S. aureus in an incubator shaker at 37° C. for 2 h. The catheters were rinsed by dipping in PBS and then incubated in a solution of compound 3a for 1 hr in an incubator shaker at 37° C. After rinsing with PBS solution, fluorescence and BLI images were acquired. Catheters were then pressed into a 4% agarose gel phantom and axial photoacoustic images acquired. As controls, sterile catheters only incubated in the S. aureus solution as well as sterile catheters which were only incubated with the probe solution were imaged to assess any autofluorescence or non-specific binding of the probe respectively.

Fluorescence and BLI signals were observed on the catheters that were incubated with S. aureus solution followed by compound 3a (FIG. 5A, left). Minimal fluorescence signal from the control catheters that do not contain biofilms was observed, illustrating low non-specific binding of the probe to the catheter (FIGS. 5A and 5B, middle). Similarly, the catheters that were incubated with S. aureus solution only showed BLI signal of the formed biofilm and no fluorescence signal (FIG. 5A, right). Axial photoacoustic images showed noticeable photoacoustic signaling on the surface of catheters that contained biofilms and incubated with compound 3a (FIG. 5C, left). Minimal photoacoustic signaling was observed on sterile catheters incubated only in compound 3a (FIG. 5D, right).

Accordingly, the present disclosure encompasses embodiments of a maltotriose-based infection imaging agent functionalized with an optical dye at the anomeric carbon. In vitro evaluation showed the efficacy and specificity of the probe to be taken up by a variety of metabolically active Gram-positive and negative bacteria. In vivo assessments showed superior performance of the maltotriose-based probe compared to its maltohexose analogue in E. coli-induced myositis murine model. Photoacoustic imaging of a bacterial infection was conducted and specifically detected the uptake of the probe in E. coli-infected thigh muscle.

Evaluations in a S. aureus-infected wound model showed the ability of the probes of the disclosure to detect and differentiate between wounds infected with different amounts of CFUs as well as to determine the effectiveness of vancomycin treatment. Finally, the utility of this the probes of the disclosure in differentiating between S. aureus-infected and sterile catheters was demonstrated using both fluorescence and photoacoustic imaging.

### Kits

The present disclosure also provides packaged compositions or pharmaceutical compositions comprising a pharmaceutically acceptable carrier and a labeled probe of the disclosure. In certain embodiments, the packaged compositions or pharmaceutical composition includes the reaction precursors to be used to generate the labeled probe according to the present disclosure. Other packaged compositions or pharmaceutical compositions provided by the present disclosure further include material including at least one of: instructions for using the labeled probe to image a subject, or subject samples (e.g., cells or tissues), which can be used as an indicator of conditions including, but not limited to, bacterial infection.

The components listed above can be tailored to the particular biological condition (bacterial infection) to be monitored as described herein. The kit can further include appropriate buffers and reagents known in the art for administering various combinations of the components listed above to the subject. The labeled probe and carrier may be provided in solution or in lyophilized form. When the labeled probe and carrier of the kit are in lyophilized form, the kit may optionally contain a sterile and physiologically acceptable reconstitution medium such as water, saline, buffered saline, and the like.

In some embodiments, the probe is provided mixed with or as a separate composition a therapeutic agent such as, but not limited to, an antibacterial agent such as an antibiotic. As will be appreciated by the skilled artisan, any combination of one or more antibiotics may be usefully be packaged in a similar manner in a kit, the antibiotic(s) being selected to target one or more bacterial infecting or colonizing a mammal or surface as described in the present disclosure may. The kit may comprise of one or two or three or more compartments. The components of the kit may be provided in separate compartments or in the same compartment. The components of the kit may be provided separately or mixed. The mixed components may contain two or more agents such as an antibiotic or a therapeutic agent for treating a pathological condition of the recipient mammal.

In some embodiments, the kit can comprise a probe according to the disclosure in a first container and can optionally further include an antibiotic, a plurality of antibiotics, a therapeutic agent or agents, and a carrier solution in one or more additional containers.

Each container of the kits will generally include at least one vial, test tube, flask, bottle, syringe or other container, into which the probe and optionally the antibiotic, therapeutic agent, carrier, and the like may be placed or suitably aliquoted. In some embodiments, the kit may comprise a suitable syringe or container for administering probe and other agents to a recipient mammal or surface.

One aspect of the disclosure encompasses embodiments of a probe comprising an oligosaccharide selectively taken up by a bacterial population and not by a mammalian cell, wherein the oligosaccharide can be connected to a detectable label by a linker and having the formula:

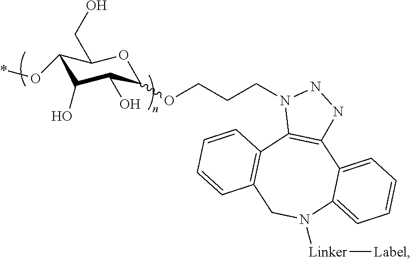

wherein n=1-8.

In some embodiments of this aspect of the disclosure, n=3 and the oligosaccharide can be a maltotriose, the probe having the formula I:

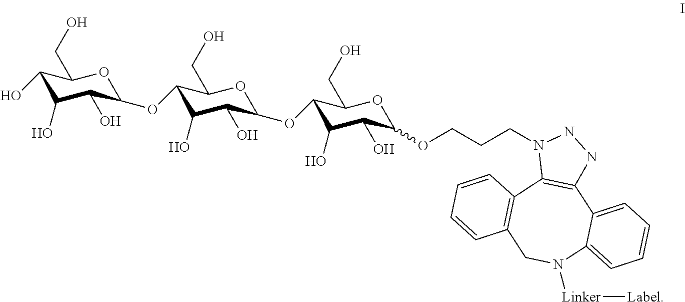

In some embodiments of this aspect of the disclosure, the detectable label can be a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

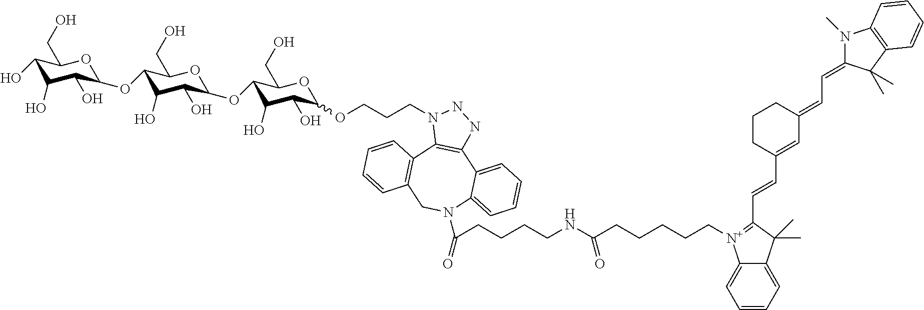

Another aspect of the disclosure encompasses embodiments of a composition comprising a probe, wherein the probe can comprise an oligosaccharide selectively taken up by a bacterial population and not by a mammalian cell and connected to a detectable label by a linker, and having the formula:

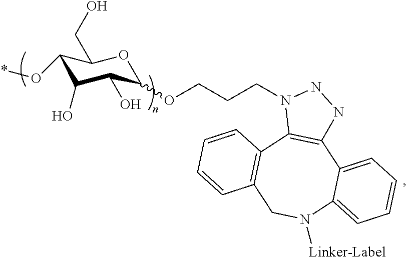

wherein n=1-8; and a pharmaceutically acceptable carrier.

In some embodiments of this aspect of the disclosure, n=3 and the oligosaccharide can be a maltotriose, the probe having the formula I:

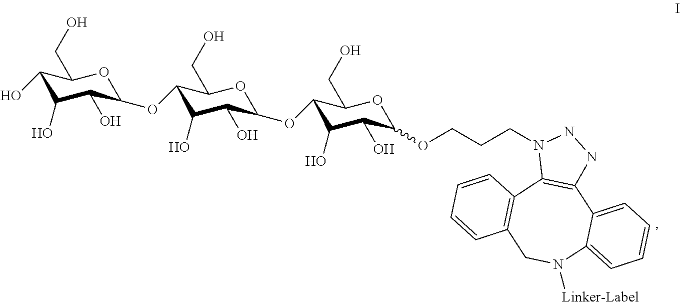

In some embodiments of this aspect of the disclosure, the detectable label is a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

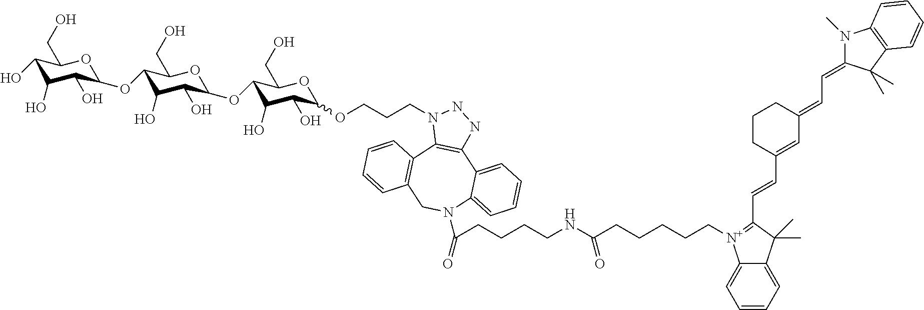

In some embodiments of this aspect of the disclosure, the composition can further comprise a therapeutic agent.

In some embodiments of this aspect of the disclosure, the therapeutic agent can be an anti-bacterial agent.

Yet another aspect of the disclosure encompasses embodiments of a method of imaging a bacterial population comprising: (i) contacting a suspected bacterial population with a composition comprising a probe, wherein the probe comprises an oligosaccharide selectively taken up by a bacterial population and not by a mammalian cell and connected to a detectable label by a linker and having the formula:

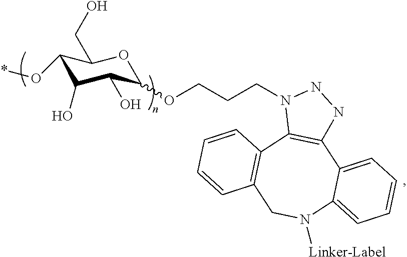

wherein n=1-8; (ii) imaging at least a portion of the subject; and (iii) detecting the labeled probe, wherein the location of the labeled probe corresponds to a bacterial population.

In some embodiments of this aspect of the disclosure, n=3 and the oligosaccharide can be a maltotriose, the probe having the formula I:

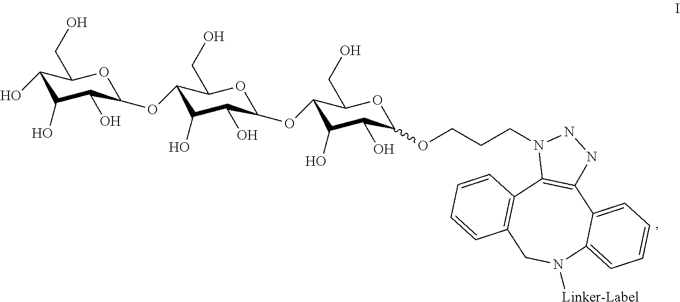

In some embodiments of this aspect of the disclosure, the detectable label can be a fluorescent dye.

In some embodiments of this aspect of the disclosure, the detectable label can be detectable photoacoustically.

In some embodiments of this aspect of the disclosure, the linker can comprise at least one oxoalkyl-amino moiety or at least one polyethylene glycol moiety.

In some embodiments of this aspect of the disclosure, the linker can be a 6-oxohexyl amino-6-oxohexylamino moiety or at least one polyethylene glycol moiety.

In some embodiments of this aspect of the disclosure, the labeled probe can have the formula:

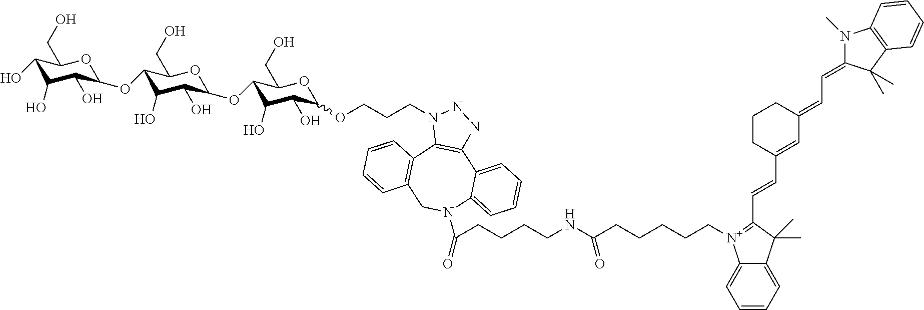

In some embodiments of this aspect of the disclosure, the method can further comprise repeating the steps (i)-(iii) periodically to monitor the progress of a bacterial infection or colonization.

In some embodiments of this aspect of the disclosure, the probe can be detected by the detection of a fluorescence signal emitted by the probe.

In some embodiments of this aspect of the disclosure, the probe can be detected by the detection of a photoacoustic signal emitted by the probe.

In some embodiments of this aspect of the disclosure, the bacterial population can be an infection of a human or animal subject.

In some embodiments of this aspect of the disclosure, the bacterial population can be a bacterial colonization of a surface.

In some embodiments of this aspect of the disclosure, the surface can be that of a surgical instrument.

In some embodiments of this aspect of the disclosure, the probe can be co-administered to the recipient subject with at least one therapeutic agent.

In some embodiments of this aspect of the disclosure, the probe can be administered to the recipient subject before administering at least one therapeutic agent.

In some embodiments of this aspect of the disclosure, the probe can be administered to the recipient subject with at least one therapeutic agent, wherein the at least one therapeutic agent is an antibiotic.

In some embodiments of this aspect of the disclosure, the method can further comprising the step of generating a series of images over a period of time, thereby indicating if the bacterial population changes in size.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### EXAMPLES

### Example 1

Chemicals were purchased from Sigma Aldrich (USA), Biosynth chemistry and biology (Switzerland), Thermo Fisher Scientific (USA) and LumiProbe (USA) with no further purification. HPLC purification was performed on a Dionex HPLC system (Dionex Corporation, Sunnyvale, Calif.) equipped with an Ultimate 3000 Pump and Ultimate 3000 RS Variable Wavelength Detector monitoring at 280 and 700 nm wavelengths. Semipreparative HPLC reverse phase column (Phenomenex, Gemini, Hesperia, Calif., C18, 5 μm, 10×250 mm) eluted at a flow rate of 3 mL/min.

### HPLC Method:

Solvent A=0.1% trifluoroacetic acid (TFA) in water; Solvent B=0.1% TFA in acetonitrile: gradient elution, 10% B (0-2 min), 10-100% B (2-20 min), 100% B (20-23 min), 10% B (23-24 min), 10% B (24-26 min). Flash chromatography was conducted on a CombiFlash® Rf+ Lumen system (Teledyne ISCO Inc., USA) equipped with an Evaporative Light Scattering Detector (ELSD detector) and a RediSep Rf Normal-phase Silica gel column (4 gm and 20 gm).

### CombiFlash Method:

Solvent A=Hexane, Solvent B=Ethyl Acetate; 0-40% B (0-5 min), 40-45% B (5-27 min), 70% B (27-35 min). ¹H and ¹³C NMR spectra were performed on an Agilent 400-MR NMR Spectrometer. Electron spray ionization (ESI) mass spectrometry was performed on a Micromass ZQ single quadrupole LC-MS system. Absorption and emission spectra collected on a TECAN SPARK plater reader. Absorbance chromatogram developed from scans collected from 500 to 1000 nm with 1 nm step size while emission chromatogram produced from scanning from 755 to 850 nm after excitation at 750 nm. In vivo bioluminescence imaging (BLI) was performed using the IVIS Spectrum Imaging System (PerkinElmer, Waltham, Mass., USA).

The mice were positioned in the instrument after being anesthetized with isoflurane and imaged under medium binning conditions for a suitable exposure time (up to 5 min). Images produced and analyzed using Living Image® software and data expressed as average radiance (p/s/cm²/sr). In vivo fluorescence imaging was performed using the IVIS Spectrum Imaging System (PerkinElmer, Waltham, Mass., USA). The mice were anesthetized with isoflurane and imaged in prone position under medium binning conditions for a suitable exposure time (up to 2 min). Images produced and analyzed using Living Image® software and data expressed as average Radiance Efficiency ([p/s]/[μW/cm²]).

### Example 2

### Synthesis of (3R,4R,5R,6R)-6-(acetoxymethyl)-5-(((3R,4R,5R,6R)-3,4-diacetoxy-6-(acetoxymethyl)-5-(((3R,4R,5R,6R)-3,4,5-triacetoxy-6-(acetoxymethyl)tetrahydro-2H-pyran-2-yl)oxy)tetrahydro-2H-pyran-2-yl)oxy)tetrahydro-2H-pyran-2,3,4-triyl Triacetate (1a) and (2S,3R,4R,5R,6R)-6-(acetoxymethyl)-5-(((3R,4R,5R,6R)-3,4-diacetoxy-6-(acetoxymethyl)-5-(((3R,4R,5R,6R)-3,4-diacetoxy-6-(acetoxymethyl)-5-(((3R,4R,5R,6R)-3,4-diacetoxy-6-(acetoxymethyl)-5-(((3R,4R,5R,6R)-3,4-diacetoxy-6-(acetoxymethyl)-5-(((3R,4R,5R,6R)-3,4,5-triacetoxy-6-(acetoxymethyl)tetrahydro-2H-pyran-2-yl)oxy)tetrahydro-2H-pyran-2-yl)oxy)tetrahydro-2H-pyran-2-yl)oxy)tetrahydro-2H-pyran-2-yl)oxy)tetrahydro-2H-pyran-2-yl)oxy)tetrahydro-2H-pyran-2,3,4-triyl Triacetate (1b)

The reaction was modified from previously reported synthesis procedure (Ning et al., Nat. Mater. 10: 602-607 (2011)). Maltotriose (257.2 mg, 0.51 mmol) or maltohexose (500 mg, 0.51 mmol) were dissolved in pyridine (10 mL) at room temperature and purged with N₂ gas. When fully dissolved, Ac₂O (5 mL) was added and solution was mixed under inert conditions at room temperature for 72 h. Solvent was then evaporated under vacuum and the precipitate was dissolved in EtOAc (100 mL). The crude mixture was washed three times in each of Na₂CO₃ (1M aq.) (10 mL), HCl 0.1M (10 mL), and brine (10 mL)×3. The organic layer was then collected and solvent dried under vacuum. The off-white precipitate was then dissolved in DCM (2 mL), loaded on a silica gel column and purified by flash column chromatography (CombiFlash method) to afford 1a and 1b in 94% and 88% yield, respectively.

1a (C₄₀H₅₄O₂₇): ¹H NMR (400 MHz, CDCl₃): δ (ppm) 6.09 (d, 1H, J=4.0 Hz), 5.62 (d, 1H, J=8.0 Hz), 5.37 (t, 1H, J=8 Hz), 5.30-5.12 (m, 4H), 4.93 (t, 1H, J=8 Hz), 4.90-4.83 (m, 1H), 4.70 (dd, 1H, J=4 Hz and 12 Hz), 4.62-4.57 (m, 1H), 4.35-4.28 (m, 2H), 4.18-4.01 (m, 4H), 3.92-3.73 (m, 6H), 2.09 (s, 1H), 2.03-2.01 (m, 5H), 1.96-1.84 (m, 24H). ESI+ MS m/z 989.37 for [1a+Na]; ESI− MS m/z 1011.33 for [1a+FA];

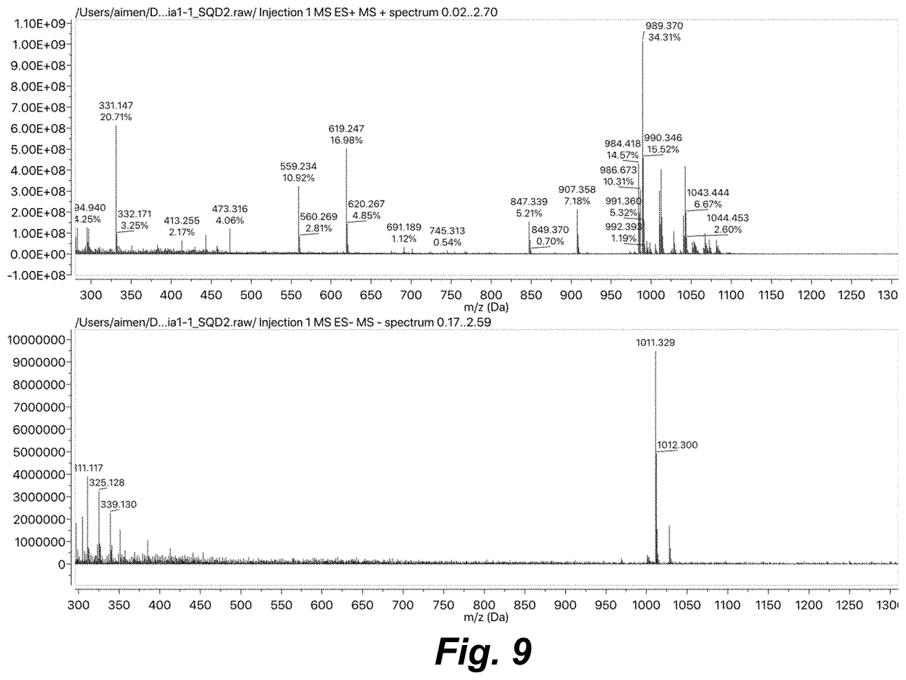

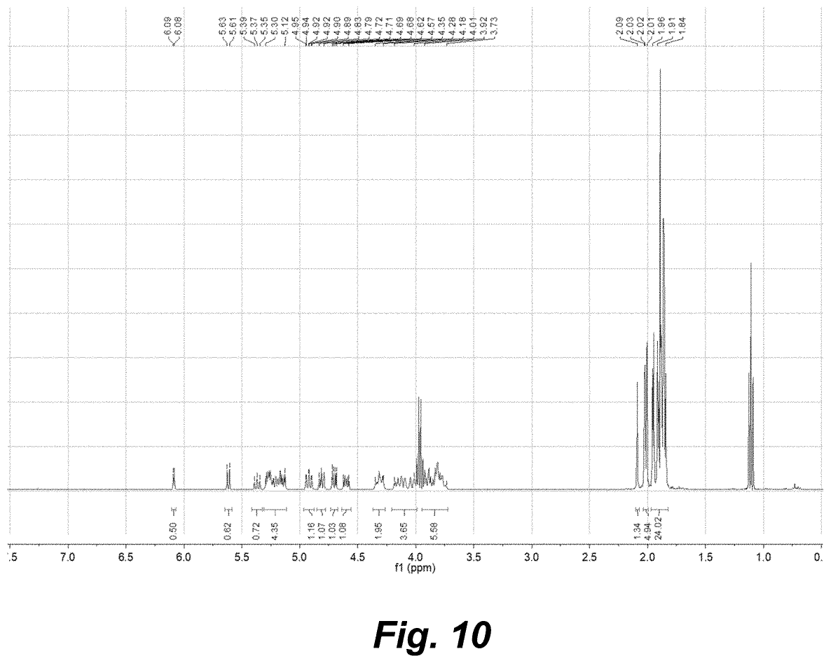

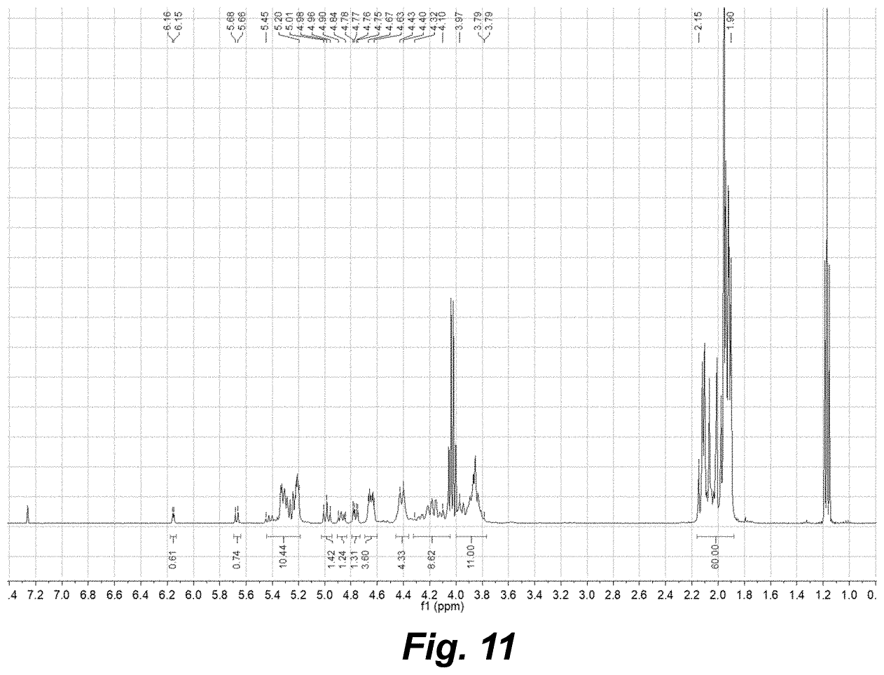

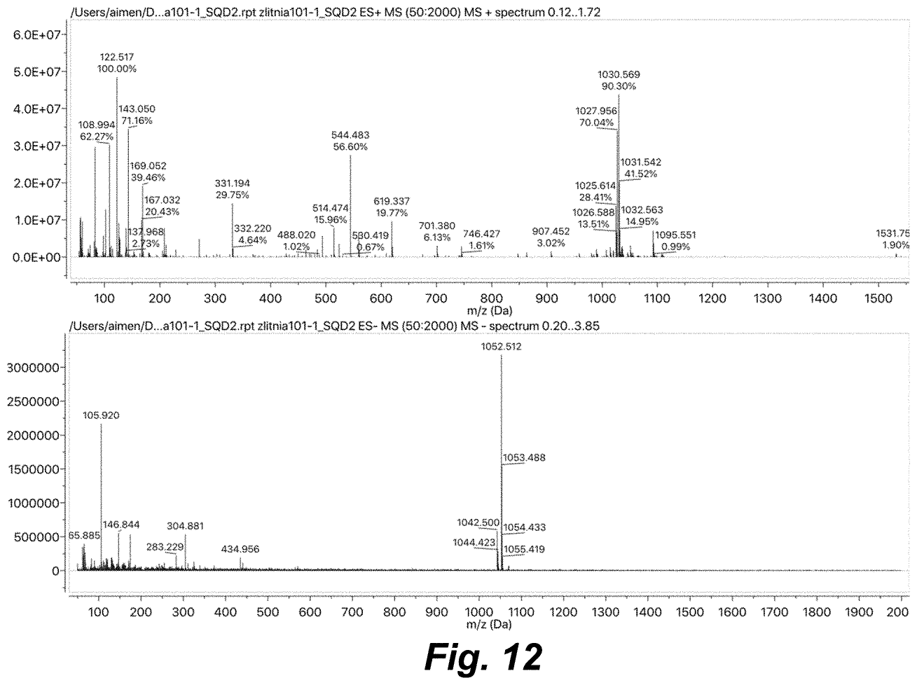

1b (O₇₆H₁₀₂O₅₁): ¹H NMR (400 MHz, CDCl₃): δ (ppm) 6.16 (d, 1H, J=4.0 Hz), 5.67 (d, 1H, J=8.0 Hz), 5.45-5.20 (m, 10H), 4.98 (t, 2H, J=12 Hz), 4.90-4.84 (m, 1H), 4.76 (dd, 1H, J=4 Hz and 8 Hz), 4.67-4.63 (m, 4H), 4.41 (d, 4H, J=12 Hz), 4.32-4.10 (m, 9H), 3.97-3.79 (m, 11H), 2.15-1.90 (m, 60H). ESI+ MS m/z 1853.54 for [1b+Na], FIGS. 9-11).

### Example 3

### Synthesis of (2R,3R,4R,5R)-2-(acetoxymethyl)-6-(((2R,3R,4R,5R)-4,5-diacetoxy-2-(acetoxymethyl)-6-(((2R,3R,4R,5R)-4,5-diacetoxy-2-(acetoxymethyl)-6-(3-azidopropoxy)tetrahydro-2H-pyran-3-yl)oxy)tetrahydro-2H-pyran-3-yl)oxy)tetrahydro-2H-pyran-3,4,5-triyl Triacetate (2a) and (2R,3R,4R,5R)-2-(acetoxymethyl)-6-(((2R,3R,4R,5R)-4,5-diacetoxy-2-(acetoxymethyl)-6-(((2R,3R,4R,5R)-4,5-diacetoxy-2-(acetoxymethyl)-6-(((2R,3R,4R,5R)-4,5-diacetoxy-2-(acetoxymethyl)-6-(((2R,3R,4R,5R)-4,5-diacetoxy-2-(acetoxymethyl)-6-(((2R,3R,4R,5R,6R)-4,5-diacetoxy-2-(acetoxymethyl)-6-(3-azidopropoxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3,4,5-triyl Triacetate (2b)

Compound 1a (389 mg, 0.402 mmol) or 1b (431.5 mg, 0.236 mmol) was placed in a round bottom flask and purged with N₂ for 10 min. The flask was then placed on dry ice and cooled down before adding 3-azido-1-propanol (3 eq) (112.09 μL, 1.206 mmol) and (67.8 μL, 0.708 mmol) respectively. The mixture was stirred on dry ice and under N₂ for 15 min before adding BF₃ (5 eq) (257.9 μL, 2.01 mmol) and (144.9 μL, 1.18 mmol) respectively. The reaction was stirred for another 2 h on dry ice and left to warm to room temperature and stirred overnight. The mixture was then quenched by adding TEA (5 eq) (282.1 μL, 2.01 mmol) and (165.6 μL, 1.18 mmol) respectively and solvent removed under vacuum. The precipitate was then dissolved in EtOAc, washed three times with brine and purified by flash chromatography (Method 2). Compounds 2a and 2b were achieved in 73% and 80% yield as an off-white precipitates.

2a (C₄₁H₅₇N₃O₂₆): ¹H NMR (400 MHz, CDCl₃): δ (ppm) 5.40-5.22 (m, 5H). 5.05 (t, 1H, J=8.0 Hz), 4.85-4.77 (m, 2H), 4.72 (dd, 1H, J=4 Hz and 8 Hz), 4.51 (d, 1H, J=8 Hz), 4.48-4.42 (m, 2H), 4.31-4.15 (m, 4H), 4.04 (dd, 1H, J=4 Hz and 12 Hz), 3.99-3.88 (m, 5H), 3.72-3.67 (m, 1H), 3.61-3.56 (m, 1H), 3.37-3.30 (m, 2H), 2.15-1.97 (m, 30H), 1.88-1.76 (m, 2H). ESI+ MS m/z 1030.57 for [2a+Na]; ESI− MS m/z 1052.51 for [2a+FA]

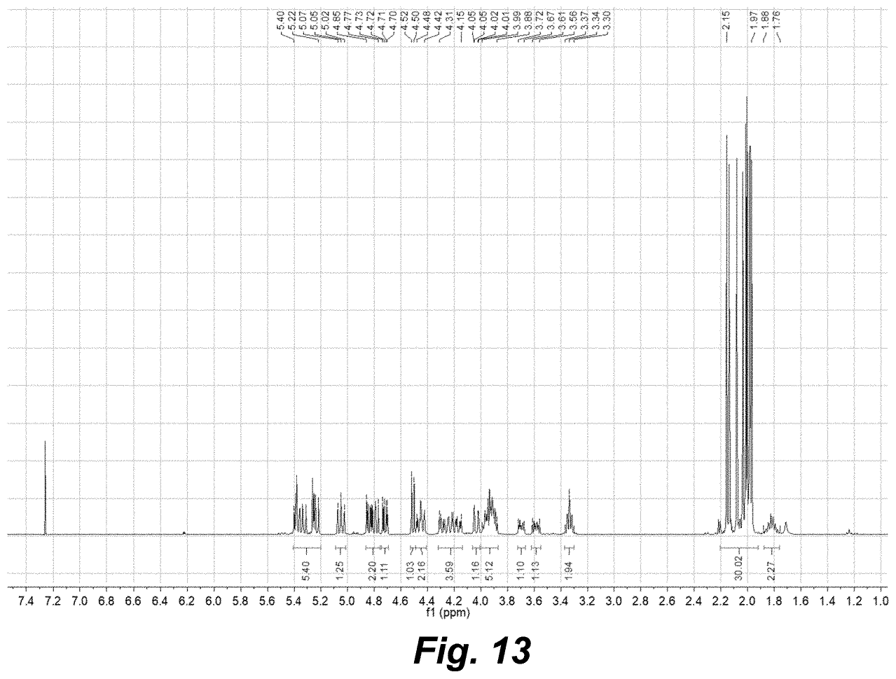

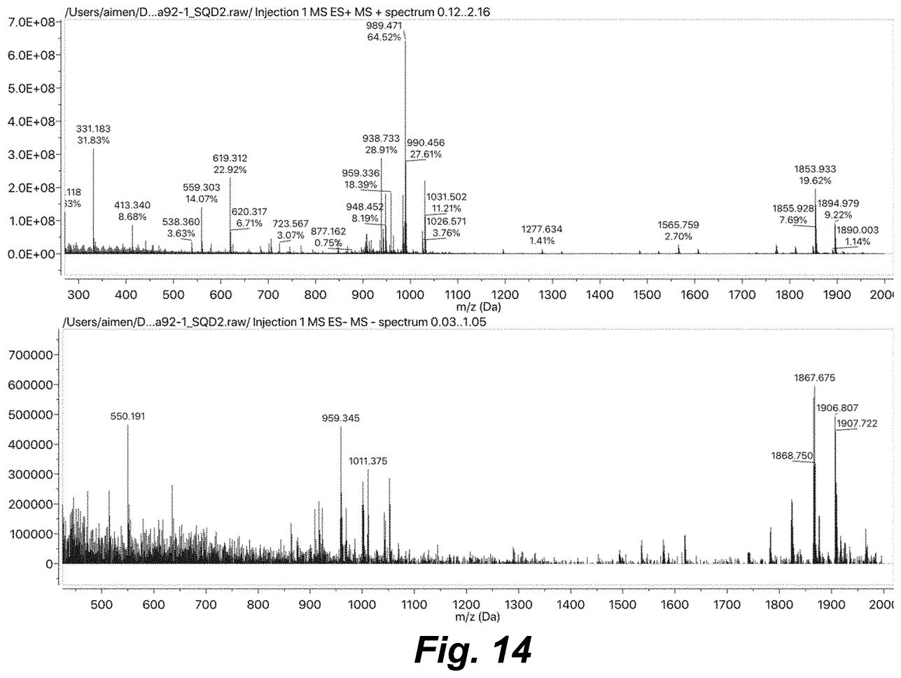

2b (O₇₆H₁₀₂O₅₁) ESI+m/z 1894.98 for [2b+Na]; ESI− MS m/z 1907.72 for [2a+CI]; (FIGS. 13-15)

### Example 4

### Synthesis of 1-(6-((6-(1-(3-(((3R,4S,5S,6R)-5-(((3R,4S,5S,6R)-3,4-dihydroxy-6-(hydroxymethyl)-5-(((3R,4R,5S,6R)-3,4,5-trihydroxy-6-(hydroxymethyl)tetrahydro-2H-pyran-2-yl)oxy)tetrahydro-2H-pyran-2-yl)oxy)-3,4-dihydroxy-6-(hydroxymethyl)tetrahydro-2H-pyran-2-yl)oxy)propyl)-1,9-dihydro-8H-dibenzo[b,f][1,2,3]triazolo[4,5-d]azocin-8-yl)-6-oxohexyl)amino)-6-oxohexyl)-3,3-dimethyl-2-((E)-2-((E)-3-(2-((E)-1,3,3-trimethylindolin-2-ylidene)ethylidene)cyclohex-1-en-1-yl)vinyl)-3H-indol-1-ium (3a) and 1-(6-((6-(1-(3-(((2R,3R,4S,5S,6R)-5-(((3R,4S,5S,6R)-5-(((3R,4S,5S,6R)-5-(((3R,4S,5S,6R)-5-(((3R,4S,5S,6R)-3,4-dihydroxy-6-(hydroxymethyl)-5-(((3R,4R,5S,6R)-3,4,5-trihydroxy-6-(hydroxymethyl)tetrahydro-2H-pyran-2-yl)oxy)tetrahydro-2H-pyran-2-yl)oxy)-3,4-dihydroxy-6-(hydroxymethyl)tetrahydro-2H-pyran-2-yl)oxy)-3,4-dihydroxy-6-(hydroxymethyl)tetrahydro-2H-pyran-2-yl)oxy)-3,4-dihydroxy-6-(hydroxymethyl)tetrahydro-2H-pyran-2-yl)oxy)-3,4-dihydroxy-6-(hydroxymethyl)tetrahydro-2H-pyran-2-yl)oxy)propyl)-1,9-dihydro-8H-dibenzo[b,f][1,2,3]triazolo[4,5-c]azocin-8-yl)-6-oxohexyl)amino)-6-oxohexyl)-3,3-dimethyl-2-((E)-2-((E)-3-(2-((E)-1,3,3-trimethylindolin-2-ylidene)ethylidene)cyclohex-1-en-1-yl)vinyl)-3H-indol-1-ium (3b)

Compound 2a (30 mg, 0.030 mmol) or 2b (60 mg, 0.032 mmol) and Cy7-DBCO (25 mg, 0.028 mmol) was dissolved in a 1:1 mixture of DCM:MeOH (6 mL) and mixture stirred at room temperature overnight. 25 wt % in methanol of NaMeOH (2 mL) was then added to the crude mixture and stirred at room temperature for 3 hr before quenching the reaction by adding AcOH (200 μL). Solvent was evaporated under vacuum and crude product dissolved in MeOH and purified by reverse-phase HPLC (HPLC Method) resulting compound 3a and 3b in 65% and 60% overall yield respectively.

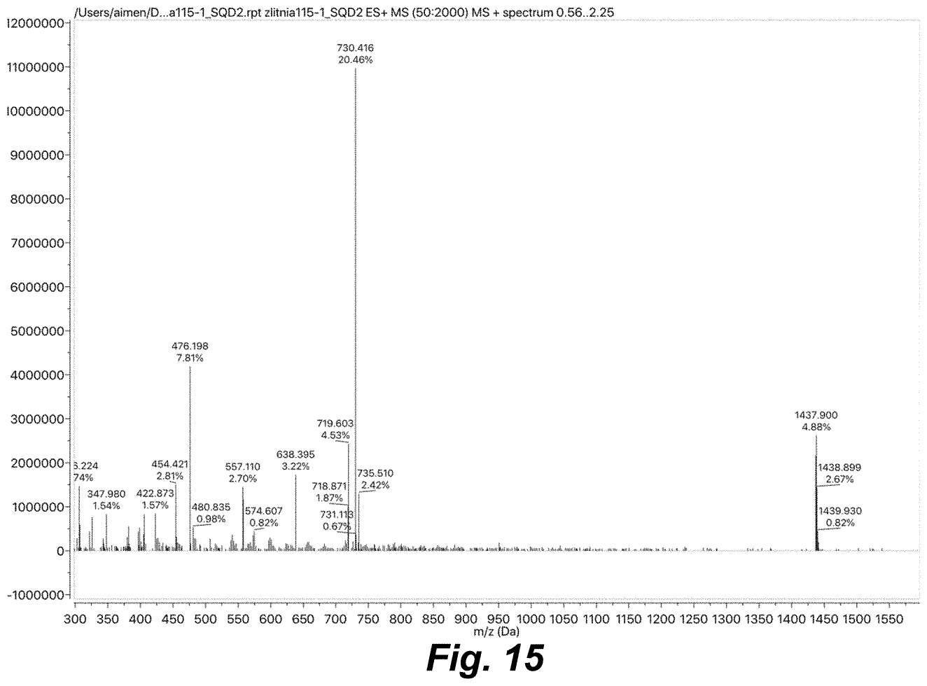

3a (C₇₉H₁₀₂N₇O₁₈): ¹H NMR (400 MHz, DMSO-d₆): δ (ppm) ESI+ MS m/z 1437.9 for [3a+H] (FIG. 15);

3b (C₉₇H₁₃₂N₇O₃₃): ¹H NMR (400 MHz, DMSO-d₆): δ (ppm) ESI+m/z 1925.2 for [3b+Na]

### Example 5

### Plasma Stability Studies:

Compound 3a or 3b (100 μM) was incubated in either PBS (1×), murine, human, or rat plasma at 37° C. for up to 24 h. At each time point (0, 2, 4, 10 and 24 h), a 100 μL aliquot of the mixture was taken and added to an ice-cold acetonitrile solution (200 μL) and vortexed for 10 seconds. Samples were then centrifuged and the supernatant analyzed on the analytical HPLC column (HPLC method). Compounds 3a and 3b had an Rf value of 15.25 and 14.65 min, respectively. Monitoring was at 750 nm and the area of any observed peak was assessed in each HPLC chromatogram. Bar plot representation of the compound stability is shown as % intact where

$$\% intact = \frac{Peak area of compound 3}{\sum Areas of all observed peaks} \times 100$$

### Example 6

### Cultures:

E. coli was obtained from American Type Culture Collections (ATCC 33456). E. coli mutants JW3992-1 (Lam B and Mal G deficient), JW3995 (Lam B and Mal K deficient) and JW1613 (Lam B and Mal X-PTS permease deficient). Bioluminescent strains of Pseudomonas aeruginosa (Xen 5), Bacillus subtilis and Staphylococcus aureus (Xen 36) were obtained from Perkin Elmer.

### Example 7

### Overnight Culture Conditions:

E. coli overnight cultures were prepared by inoculating a colony in Luria-Bertani (LB) broth (3 mL) in an incubator-shaker at 37° C. The mutant E. coli strains and Xen36, the bioluminescent strain of S. aureus, were grown in LB with kanamycin (50 μg/ml). After 16 h, 600 μL of the 0/N culture was added to 30 mL of LB in a 200 mL flask and placed in an incubator-shaker at 37° C. until bacterial culture reached the log phase (OD₆₀₀=0.5). Metabolically-inactive E. coli was prepared by either treating an overnight culture (OD₆₀₀=0.5) with sodium azide (10 mM) and incubating for 1 hr in an incubator-shaker at 37° C. or by heating the culture to 90° C. for 30 min. All cultures were harvested by centrifugation and pellets washed three times with HBSS (1×) before suspending at the concentration of interest.

### Example 8

### Bacteria Competition Assay:

Aliquots of 10⁸ colony forming units (CFU) of E. coli were first incubated with test compounds (1 mM) for 1 hr at 37° C. After the first incubation, the bacteria culture was centrifuged at 10,000 rpm for 5 min and pellet washed with 1×HBSS three times. Pellets were suspended in a solution of ³H-maltose (1 μCi in 200 μL HBSS (1×); American Radiolabeled Chemicals, Inc., USA) and incubated for 30 min at 37° C. Aliquots were centrifuged and washed with 1×HBSS three times before lysing in a bacterial lysis buffer (BugBuster, EMD, Billerica Mass. USA). Activity in bacteria lysates was then counted in a g-counter and protein concentration determined using a bicinchoninic acid (BCA) assay (Pierce, Thermo Fisher Scientific). Test compounds included maltose as a positive control, azide-1-maltotriose/maltohexose and Cy7-1-maltotriose/maltohexose. In addition, aliquots incubated with only ³H-maltose were also included to assess normal uptake. Results are shown as counts per minute (cpm) normalized to protein content (μg of protein) per sample (n=3 per study).

### Example 9

### Bacteria Uptake Studies:

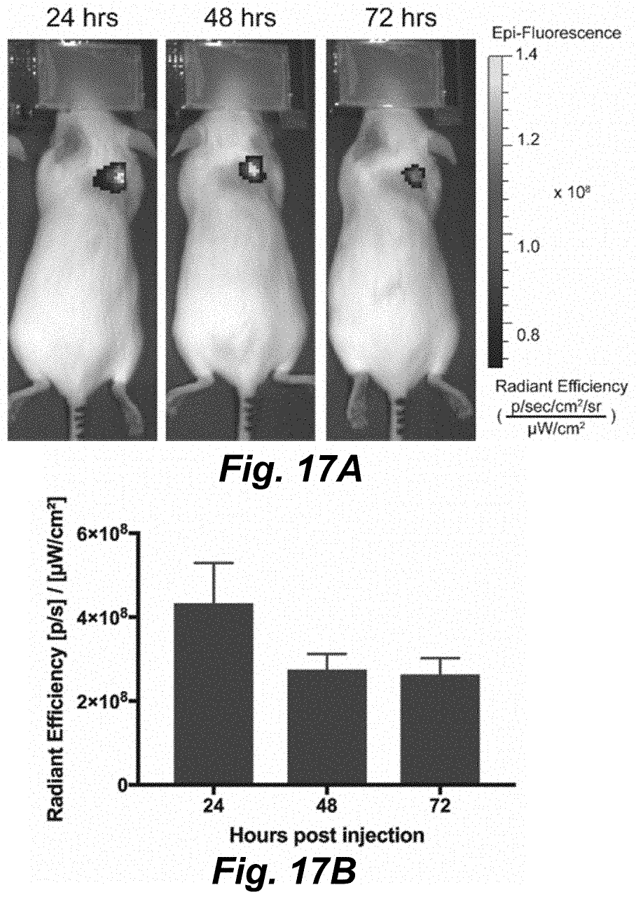

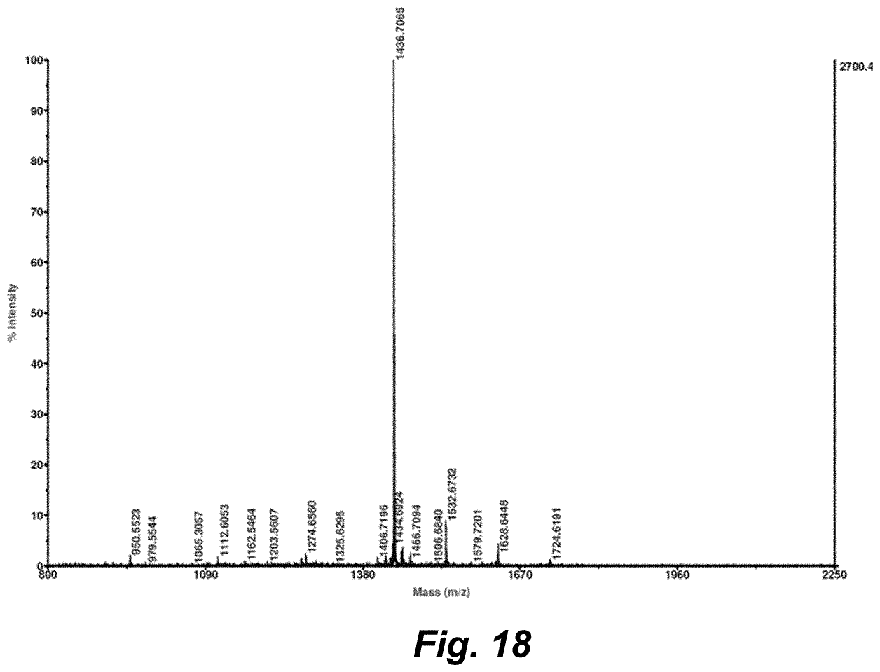

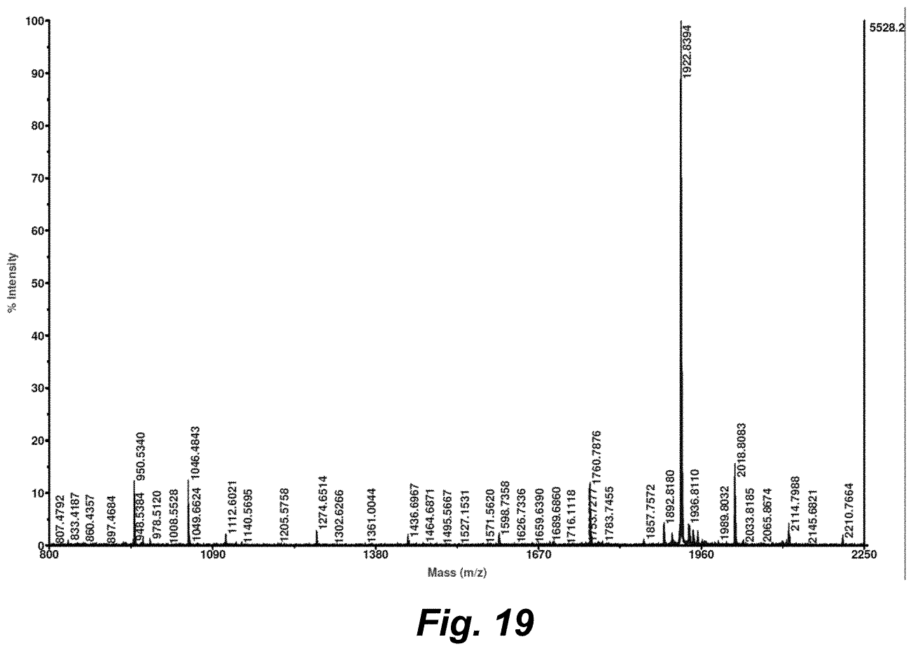

Aliquots of 10⁸ colony forming units (CFU) of E. coli, E. coli mutants, azide-inactivated E. coli, Pseudomonas aeruginosa, Bacillus subtilis and Staphylococcus aureus (Xen 36) were incubated with the same amount of compound 3a for 1 hr at 37° C. Aliquots were then centrifuged (10,000 rpm for 5 min) and washed with 1×HBSS three times before lysing in a bacteria lysis buffer (BugBuster® for E. coli and mutants, EMD; B-PER® Complete Bacterial Protein Extraction Reagent for the rest of strains, Thermo Scientific®). The fluorescence intensity in bacteria lysates was measured in a SpectraMax GEMINI EM fluorescent plate reader (Molecular Devices, USA) (FIG. 17).

### Example 10

### In Vitro Influx Studies:

Aliquots of 10⁸ colony forming units (CFU) of E. coli were incubated with the same amount of compound 3a or 3b (50 μM) at 37° C. After each time point (30, 60, 240 and 1080 min), aliquots were then centrifuged (10,000 rpm for 5 min) and washed with HBSS (1×) three times before lysing in a bacteria lysis buffer (BugBuster for E. coli and mutants, EMD; B-PER® Complete Bacterial Protein Extraction Reagent for the rest of strains, Thermo Scientific®). The fluorescence intensity in bacteria lysates was measured in a SpectraMax GEMINI EM fluorescent plate reader (Molecular Devices, San Jose, Calif., USA).

### Example 11

### Animals and Infection Models

E. coli-Induced Murine Myositis:

Female nu/nu mice, 6-7 weeks old were anesthetized by isoflurane inhalation (2-3%). 10⁸ CFU of E. coli in 50 μL of 1×HBSS was injected intramuscularly into the right thigh muscle of the mice. As a control, 10⁸ CFU of heat-inactivated E. coli or E. coli MaIG+LamB was injected intramuscularly in the left thigh.

Staphylococcus aureus Wound-Infection Murine Model:

Female CD1 or SKH1 elite mice, 6-8 weeks old were anesthetized by isoflurane inhalation (2-3%). A small wound on the upper or lower back of the mice was formed using a sharp pair of scissors. 10⁸ CFU, 10⁶ CFU, or 10⁴ CFU Staphylococcus aureus in 20 μL saline was inoculated into a small pocket subcutaneously before sealing the wound with Vetbond adhesive (1469SB; 3M).

### Example 12

In Vivo Imaging in E. coli-Induced Myositis Model

### In Vivo Distribution Overtime:

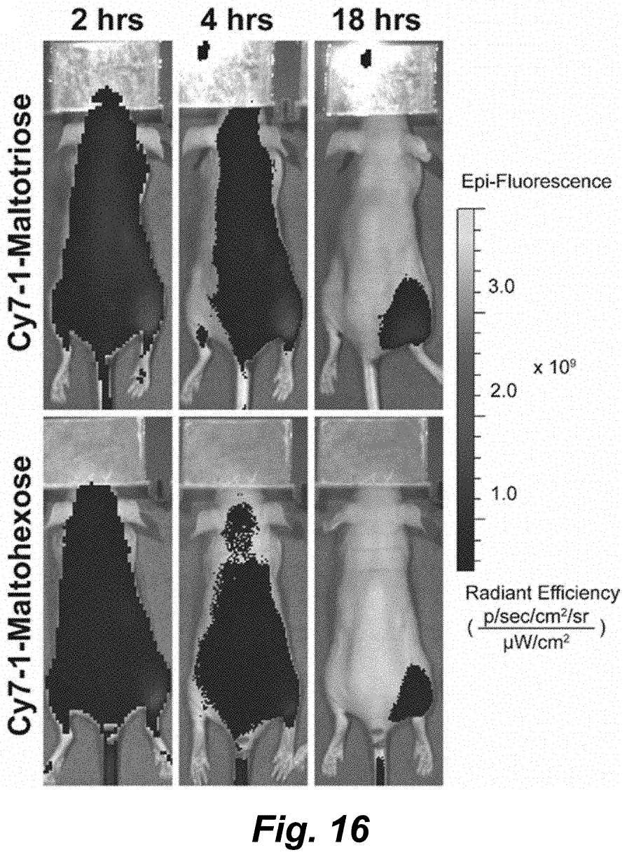

Directly after E. coli-induced murine myositis (n=4), Cy7-1-maltotriose (5 nmol in 2% DMSO/saline, 100 μL) was injected via the tail vein. Fluorescence images were captured using an IVIS Spectrum Imaging System (PerkinElmer, USA) at 1 h, 3 h, 5 h and 20 h post injection of the agent. The fluorescence intensity in the right thigh muscle (E. coli) and left thigh muscle (heat-inactivated E. coli) were quantified and analyzed. In vivo comparison to maltohexose derivative: Directly after E. coli-induced murine myositis, Cy7-1-maltotriose (compound 3a, n=6) or Cy7-1-maltohexose (compound 3b, n=4) (5 nmol in 2% DMSO/Saline, 100 μL) were injected via the tail vein. Fluorescence images (FIG. 16) were captured using an IVIS Spectrum Imaging System (PerkinElmer, USA) at 2 h, 4 hr and 18 hr post injection of the agent. The fluorescence intensity in the right thigh muscle (E. coli) and left thigh muscle (control) were integrated. Data was presented as the ratio of fluorescence intensity in the right versus left thigh muscle (infected:control muscle ratio) or fluorescence intensity in the right thigh muscle versus whole body fluorescence (% fluorescence) (as in FIG. 4B). Image analysis was conducted using Living Image® software.

### Example 13

### Photoacoustic Imaging:

20 hr and 25 hr post injection of Cy7-1-maltotriose (before and after treatment respectively), mice were anesthetized and fixed in the prone position for photoacoustic imaging. PA and US images were then collected using a Vevo3100 LAZR imaging system (Vevo® LAZR, Visual Sonics, Inc., Canada) with PA-mode and B-mode respectively. The PA system was equipped with a MX-250 transducer and irradiation occurred under a 680, 700, 750 and 800 nm laser. A 3D scan image of the wound's site was acquired, and 3D rendered images from the 700 nm irradiation scan presented as photoacoustic image overlaid on ultrasound image were produced using VevoLAB software (Visual Sonics, Inc., Canada). The whole region of the wound was highlighted using VevoLAB software and average PA signal intensity quantified and presented as arbitrary units (a.u.).

### Example 14

### Bacterial Burden Differentiation (CD1 Mouse Model):

After subcutaneous inoculation of 10⁸ CFU (n=5), 10⁶ CFU (n=3) and 10⁴ CFU (n=3) of S. aureus in the wound, Cy7-1-maltotriose (5 nmol in 2% DMSO/saline, 100 μL injection) was injected via the tail vein. To insure no other infections occur, a daily dose of kanamycin (800 mg/kg) was given to the mice intramuscularly. Fluorescence and bioluminescence images were then captured at 5 and 20 hr post injection of the probe. Image analysis was conducted using Living Image® software.

### Example 15

### In Vivo Specificity of Cy7-1-Maltotriose to Bacteria Containing Maltodextrin Transporter:

Myositis in mouse was induced by injecting 10⁸ CFU of E. coli and 108 CFU of E. coli MaIG+LamB mutant in the right and left thigh respectively (n=5). Cy7-1-maltotriose (10 nmol in 2% DMSO/saline, 200 μL injection) was then injected via the tail vein. Fluorescence images were acquired 3 and 20 hr post injection. After imaging, mice were sacrificed both right and left thigh muscle collected and ex-vivo fluorescence images acquired.

### Example 16

### Treatment Study (SKH1-Elite Mouse Model)

Fluorescence and Bioluminescence Imaging: After subcutaneous inoculation of 10⁶ CFU (n=9) of kanamycin-resistant S. aureus in the wound, mice were administered kanamycin (800 mg/kg) intramuscularly once daily to insure no other bacterial infections occur. Two days after surgery, Cy7-1-maltotriose (10 nmol in 2% DMSO/saline, 200 μL injection) was injected via the tail vein and bioluminescence and fluorescence imaging were performed at 19, 45, 69, 93 and 144 hrs post injection. Following imaging at 19 h, the mice were divided into untreated (n=4) and treated (n=5) groups where the first only received kanamycin and the latter were administered vancomycin (110 mg/kg) subcutaneously twice daily in addition to kanamycin. After 7 days of antibiotic treatment, mice were injected again with Cy7-1-maltotriose (10 nmol in 2% DMSO/saline, 200 μL injection) and bioluminescence and fluorescence imaging performed 24 hr post injection.

### Example 17

In Vitro Imaging of S. aureus-Infected Biomaterial:

Sterile catheters were collected from a BD Insyte® Autoguard® BC Shielded IV Catheter (ref: 382544, BD, USA). Catheters were then placed in a 10⁶ CFU of S. aureus/mL solution in an incubator shaker at 37° C. for 2 h. Catheters were then placed in a Cy7-1-maltotriose solution (50 nmol/mL) and incubated for 37° C. for 1 hr before washing by gently dipping in PBS (1×) solution. As a control, sterile catheter that were not exposed to infection or infected catheters that were not incubated with the probe were assessed. BLI and fluorescence images of the catheters were then collected using an IVIS Spectrum Imaging System (PerkinElmer, USA). Image analysis was then conducted using Living Image® software. In addition, the catheters were placed inside a 2% agarose phantom and axial ultrasound and photoacoustic images collected on a Vevo LAZR imaging system (Vevo® LAZR, Visual Sonics, Inc., Canada). Photoacoustic and US images were analyzed using Vevo LAB software (Visual Sonics, Inc., Canada).

### Example 18

In Vivo Evaluation in E. coli-Induced Myositis Murine Model:

Preclinical evaluation of Cy7-1-maltotriose in relevant murine models (E. coli-induced myositis and S. aureus-infected wound) was then conducted. Fluorescence imaging was used to assess the specificity and uptake of the probe in bacterial infections because it allows whole-mouse imaging and provides information that can be compared to previous reports on optical infection imaging agents. In addition, photoacoustic imaging was used to visualize the uptake of the probe in bacterial infections.

Upon intravenous injection of the probe Cy7-1-maltotriose of the disclosure into mice with E. coli-induced myositis (right thigh muscle), whole-body fluorescence images overtime were acquired (1, 3, 5 and 20 h). As a control, the mice were also injected (left thigh muscle) with the same amount of heat-inactivated E. coli demonstrates the specificity of this probe for metabolically active bacteria.

Fluorescence images show clearance of the probe though the kidneys with residual circulating probe for up to 5 h. The overnight image (20 h) showed a signal to noise ratio of about 3:1. Even the 1 hr imaging time point presented a significantly higher fluorescence signal in the infected muscle (right thigh) compared to control muscle (left thigh) (FIG. 3B, P<0.0001). Photoacoustic images of the thigh muscle also showed a higher signal in the infected muscle (right thigh) than control muscle (left thigh) (FIG. 3C). In addition to imaging the control muscle, images of both the infected and control thigh muscle were acquired before injection of the probe to assess any intrinsic background signal. The quantified intrinsic background photoacoustic signal was significantly lower than the photoacoustic signal in the infected thigh muscle post probe-injection (FIG. 3D, P=0.0001).

A comparison was later conducted between the maltotriose and maltohexose derivative in the same myositis model. Equal amounts of Cy7-1-maltotriose and Cy7-1-maltohexose were injected via the tail vein of mice with E. coli-induced myositis model (n=6 and 4 respectively). Both probes showed specific uptake by the infected muscle (right thigh) that is distinguishable from the control muscle (left thigh) (FIG. 3E). Eighteen hour post-injection, images showed a 2.6× higher total fluorescence signal in the infected thigh muscle when injecting Cy7-1-maltotriose compared to Cy7-1-maltohexose (15.5±5.0×10⁹ and 6.02±0.5×10⁹ Radiance efficiency, respectively). However, the fluorescence images and quantification overtime (2, 4 and 18 hr post injection) did show faster clearance of the Cy7-1-maltohexose compared to Cy7-1-maltotriose (FIG. 5A). This suggests differences in in vivo pharmacokinetics between the two probes.

The signal in the infected muscle (right thigh) was normalized to whole body fluorescence or to signal in the control muscle (left thigh) for proper comparison. The signal normalized to whole-body (% fluorescence) as well as the ratio of signal in the infected over control muscle (ratio) indicated superior in vivo performance of Cy7-1-maltotriose compared to Cy7-1-maltohexose (FIGS. 3F and 5B). In vitro influx studies were used to assess the probes uptake in E. coli over time (at 30, 60, 240 and 1080 min).

After 30 min of incubation, significantly higher fluorescence signal when incubating with Cy7-1-maltotriose compared to Cy7-1-maltohexose was observed. In addition, both probe's uptake reached saturation by the 60 min incubation time point. These observations suggest a faster uptake of the maltotriose derivative compared to maltohexose. The higher hydrophilicity of the maltohexose derivative could be causing its faster in vivo clearance though the kidneys (C Log P=−5.8 vs −12.3 for maltotriose and maltohexose respectively). The slower bacterial-uptake of the maltohexose derivative, its faster clearance due to its hydrophilicity and its previously reported lower binding affinity to MBP (Axer et al., ChemMedChem 13: (2017)) support that Cy7-1-maltotriose is an advantageous bacterial-imaging probe.

### Example 19

In Vivo Evaluation in S. aureus-Infected Wound Murine Model:

The probes of the disclosure are advantageous for the detection and monitoring of treatment of bacterial infections in susceptible sites (i.e. wounds, surgical sites and medical implants). Accordingly, the efficacy and sensitivity of Cy7-1-maltotriose was assessed with fluorescence imaging in detecting and monitoring treatment of a S. aureus-infected wound in a murine model.

S. aureus is the most common cause of surgical site infections and results in a 5% increase in mortality (Anderson & Kaye Infect. Dis. Clin. North Am. 23: 53-72 (2009)). S. aureus strain Xen 36, is kanamycin resistant and produces its own substrate allowing detection though bioluminescence imaging without the need to inject any substrate. During the studies, the mice were administered a dose of kanamycin once daily to insure no other bacterial contaminations occur. An increase in fluorescence signal (i.e. indicative of Cy7-1-maltotriose accumulation) was observed with an increase in CFU of S. aureus in the wound (i.e. increase in BLI signal).

Wounds infected with as low as 10⁴ CFU were detectable by fluorescence imaging of Cy7-1-maltotriose and showed great correlation with the location of the BLI signal (FIGS. 4A and 4B). A treatment study initiated in mice with wound infected with S. aureus showed that after 16 days of treatment and 24 hr post injection of Cy7-1-maltotriose, no fluorescence nor BLI signal were observed in the wound (FIG. 4C). In addition, a significant decrease in the fluorescence signal after treatment was observed compared to signal before initiating the treatment regimen (FIG. 4D, P<0.0001). Both the increase in Cy7-1-maltotriose uptake in the wound with increase in CFU of S. aureus (P<0.0114) and its decrease in uptake after antibiotic treatment show the effectiveness of this probe accompanied with the proper imaging modality to non-invasively assess bacterial burden in infected sites and determine the success of treatment regimen. The fluorescence signal coming from Cy7-1-maltotriose when taken up by S. aureus was detectable and differentiated from surrounded tissue for up to 72 hr post injection illustrating high retention and sustainability (and/or stability) of the probe.

### Example 20

### In Vitro Imaging of Biomaterial Infections:

Cy7-1-maltotriose was used to image S. aureus-infected catheters. S. aureus is the most prevalent pathogen in medical device infections and accounts for about 32% of medical device infections (Arciola et al., Nat. Revs. Microbiol. 16: 397-409 (2018)). Thus, sterile catheters were incubated in a culture solution containing 10⁶ CFU of S. aureus followed by a solution of Cy7-1-maltotriose. After a rinse, fluorescence and BLI images of the catheters were acquired. As a control, catheters only incubated with Cy7-1-maltotriose or S. aureus cultures were also tested and imaged.

Interestingly, BLI signal of Xen 36 was evident in the catheters incubated with S. aureus culture (FIG. 5A). In addition, infected catheters showed an intense fluorescence signal when incubated in a solution containing Cy7-1-maltotriose while sterile catheters showed minimal fluorescence signal (FIG. 5A). The same catheters were then pressed into a tissue mimicking 4% agarose gel phantom and axial photoacoustic images acquired. Similar to the fluorescence imaging results, minimal photoacoustic signal was observed on the sterile catheters while evident photoacoustic signal was seen on the infected catheters post incubation with Cy7-1-maltotriose (FIG. 5B).

What is claimed:

1. A probe comprising an oligosaccharide selectively taken up by a bacterial population and not by a mammalian cell, wherein the oligosaccharide is connected to a detectable label by a linker and having the formula: 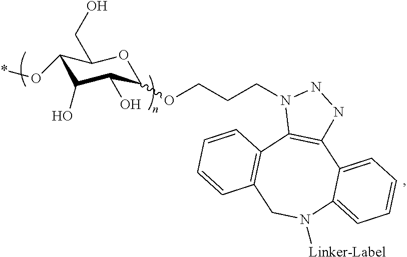
wherein n=1-8.

2. The probe of claim 1, wherein n=3 and the polysaccharide is a maltotriose, the probe having the formula I:

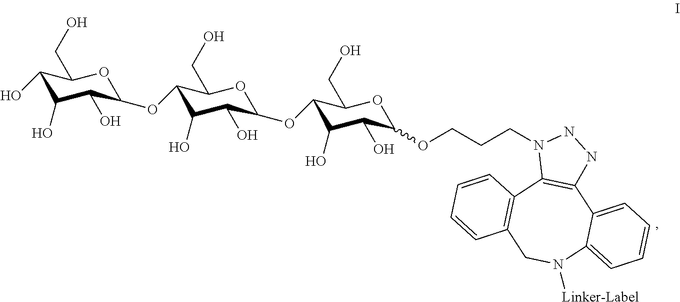

3. The probe of claim 1, wherein the detectable label is a fluorescent dye.

4. The probe of claim 1, wherein the detectable label is detectable photoacoustically.

5. The probe of claim 1, wherein the linker comprises at least one oxoalkyl-amino moiety or at least one polyethylene glycol moiety.

6. The probe of claim 1, wherein the linker is a 6-oxohexyl amino-6-oxohexylamino moiety or at least one polyethylene glycol moiety.

7. The probe of claim 1, wherein the labeled probe has the formula:

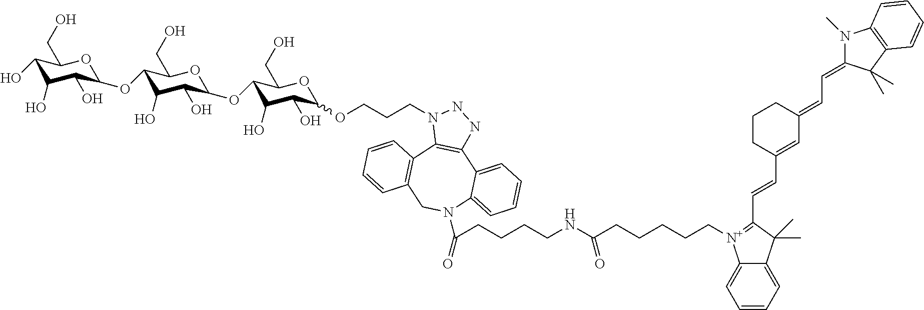

8. A composition comprising a probe, wherein the probe comprises an oligosaccharide selectively taken up by a bacterial population and not by a mammalian cell and connected to a detectable label by a linker and having the formula: 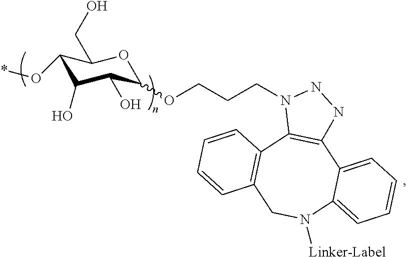
wherein n=1-8; and
a pharmaceutically acceptable carrier.

9. The composition of claim 8, wherein n=3 and the oligosaccharide is a maltotriose, the probe having the formula I:

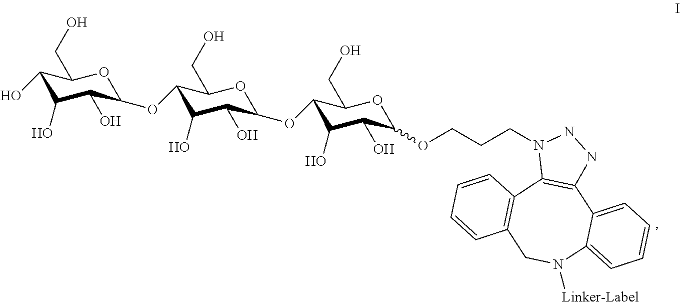

10. The composition of claim 8, wherein the detectable label is a fluorescent dye.

11. The composition of claim 8, wherein the detectable label is detectable photoacoustically.

12. The composition of claim 8, wherein the linker comprises at least one oxoalkyl-amino moiety or at least one polyethylene glycol moiety.

13. The composition of claim 8, wherein the linker is a 6-oxohexyl amino-6-oxohexylamino moiety or at least one polyethylene glycol moiety.

14. The composition of claim 8, wherein the labeled probe has the formula:

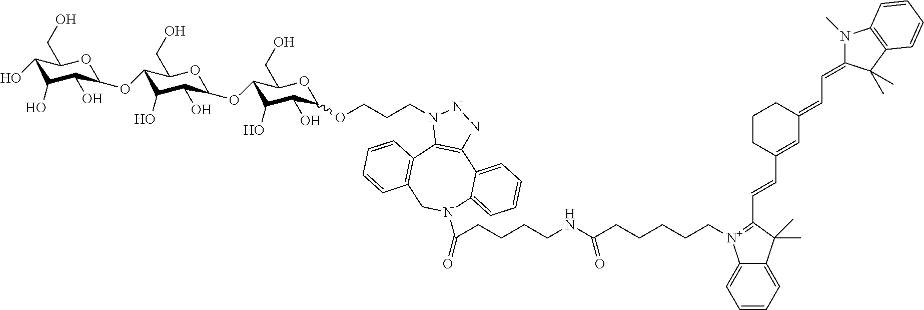

15. The composition of claim 8, wherein the composition further comprises a therapeutic agent.

16. The composition of claim 15, wherein the therapeutic agent is an anti-bacterial agent.

17. A method of imaging a bacterial population comprising:
(i) contacting a suspected bacterial population with a composition comprising a probe, wherein the probe comprises an oligosaccharide selectively taken up by a bacterial population and not by a mammalian cell and connected to a detectable label by a linker and having the formula: 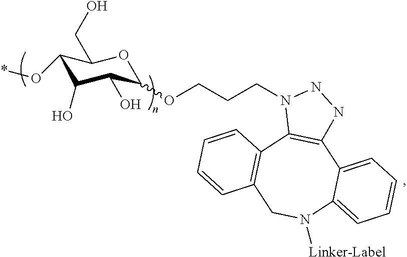
wherein n=1-8;
(ii) imaging at least a portion of the subject; and
(iii) detecting the labeled probe, wherein the location of the labeled probe corresponds to a bacterial population.

18. The method of claim 17, wherein in the probe, n=3 and the oligosaccharide is a maltotriose, the probe having the formula I:

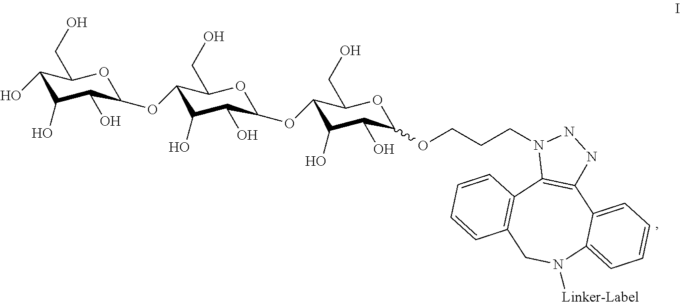

19. The method of claim 17, wherein the detectable label is a fluorescent dye.

20. The method of claim 17, wherein the detectable label is detectable photoacoustically.

21. The method of claim 17, wherein the linker comprises at least one oxoalkyl-amino moiety or at least one polyethylene glycol moiety.

22. The method of claim 17, wherein the linker is a 6-oxohexyl amino-6-oxohexylamino moiety or at least one polyethylene glycol moiety.

23. The method of claim 17, wherein the labeled probe has the formula:

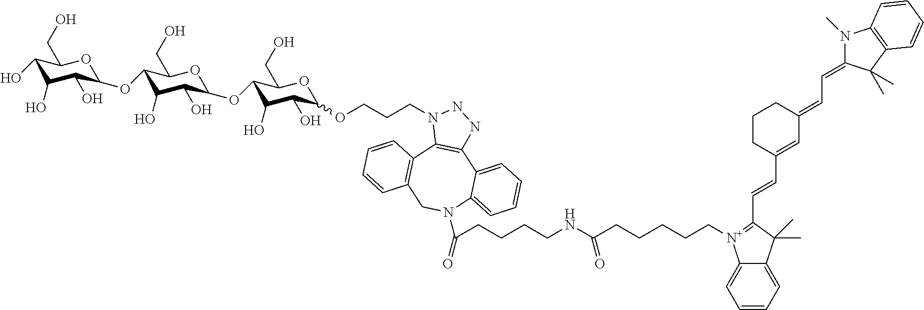

24. The method of claim 17, further comprising repeating the steps (i)-(iii) periodically to monitor the progress of a bacterial infection or colonization.

25. The method of claim 17, wherein the probe is detected by the detection of a fluorescence signal emitted by the probe.

26. The method of claim 17, wherein the probe is detected by the detection of a photoacoustic signal emitted by the probe.

27. The method of claim 17, wherein the bacterial population is an infection of a human or animal subject.

28. The method of claim 13, wherein the bacterial population is a bacterial colonization of a surface.

29. The method of claim 28, wherein the surface is that of a surgical instrument.

30. The method of claim 17, wherein the probe is co-administered to the recipient subject with at least one therapeutic agent.

31. The method of claim 17, wherein the probe is administered to the recipient subject before administering at least one therapeutic agent.

32. The method of claim 17, wherein the probe is administered to the recipient subject with at least one therapeutic agent, wherein the at least one therapeutic agent is an antibiotic.

33. The method of claim 17, further comprising the step of generating a series of images over a period of time, thereby indicating if the bacterial population changes in size.
